(19) United States
(12) Patent Application Publication
(10) Pub. No.: US 2005/0112131 A1
(43) Pub. Date: May 26, 2005

(54) Methods of adjuvant photodynamic therapy to enhance radiation sensitization

(76) Inventors: Brian Pogue, Hanover, NH (US); Julia A. O'Hara, Hanover, NH (US); Harold M. Swartz, Hanover, NH (US); Tayyaba Hasan, Boston, MA (US)

Correspondence Address: EDWARDS & ANGELL, LLP, P.O. BOX 55874, BOSTON, MA, 02205, US

(21) Appl. No.: 10/949,153

(22) Filed: Sep. 24, 2004

Related U.S. Application Data
(63) Continuation-in-part of application No. PCT/US03/09368, filed on Mar. 25, 2003.
(60) Provisional application No. 60/398,233, filed on Jul. 23, 2002.
(60) Provisional application No. 60/367,593, filed on Mar. 25, 2002.

(51) Int. Cl. A61K 39/395; A61N 1/30; A61K 31/555; A61K 31/409; A61K 31/195

(52) U.S. Cl. 424/178.1; 514/185; 514/410; 514/561; 604/20

(57) ABSTRACT

The present invention relates to the enhancement of radiation sensitivity by using photodynamic therapy.

### RELATED APPLICATIONS/PATENTS & INCORPORATION BY REFERENCE

This application is a continuation-in-part of International Application No. PCT/US03/09368, filed on Mar. 26, 2003, claiming priority to U.S. application Ser. No. 60/398,233, filed on Jul. 23, 2002, and to U.S. application Ser. No. 60/367,593, filed on Mar. 25, 2002. This application makes reference to U.S. application Ser. No. 10/137,029, filed on May 1, 2002, the contents of which are incorporated herein by reference.

### STATEMENT OF RIGHTS TO INVENTIONS MADE UNDER FEDERALLY SPONSORED RESEARCH

This work was supported by the government, in part, by grants from the National Institutes of Health (Grant No. P41 RR11602) and from the National Cancer Institute (Grant Nos. RO1CA78734 and PO1CA84203). The government may have certain rights to this invention.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference. Documents incorporated by reference into this text may be employed in the practice of the invention.

### BACKGROUND OF THE INVENTION

Photodynamic therapy (“PDT”) is a treatment modality for tissues undergoing rapid division. This therapy involves the selective uptake and retention of a photoactive agent in a cell or cells of interest, usually microbial cells, or tumor cells. These cells are then exposed to light of a particular wavelength, which excites the photoactive agent to its first excited triplet state, which is then efficiently quenched by molecular oxygen to produce singlet oxygen (Weishaupt, K., et al 1976; Henderson, B. W. and Dougherty, T. J., 1992). Singlet oxygen is highly toxic to cells and thereby initiates necrosis or apoptosis in the cell of interest.

PDT is a binary therapy, having the advantage of inherent dual selectivity. First, selectivity is achieved by an increased concentration of the photosensitizer in target tissue, and second, the irradiation can be limited to a specified volume. Provided that the photosensitizer is non-toxic, only the irradiated areas will be affected, even if the photosensitizer does bind to normal tissues. Selectivity thus obtained may be adequate for certain anatomical sites, such as skin and oral cavity, however, for more complex sites such as the peritoneal cavity, greater selectivity than that achievable with current photosensitizers is necessary, so that colateral damage to normal organs can be minimized. Selectivity can be even further enhanced by attaching photosensitizers to molecular delivery systems that have high affinity for target tissue (Hasan, 1992), (Strong et al., 1994). For example, one way to improve selectivity is to link the photosensitizer to a monoclonal antibody directed against cancer-associated antigens in an approach known as photoimmunotherapy (“PIT”). The resulting photoimmunoconjugate (“PIC”) delivers the photosensitizer directly to the tumor cell of interest.

In theory, the tumoricidal efficacy of PDT can be increased when used in combination with other anti-cancer therapies, such as ionizing radiation. However, several studies have investigated the combination effect of hematoporphyrin photosensitizers by themselves and with γ-radiation, and found only a modest, additive enhancement, or no enhancement at all (Kostron, H., 1986; Moan, J., et al 1981). Additionally, studies have also examined combined PDT treatment with radiation treatment, but only within the context of cultured cells (Luksiene, Z., et al. 1999; Ramakrishnan, N., et al. 1990). Even in cultured cells, only modest, additive effects were observed with combination PDT and radiation therapy. Studies with aluminum phthalocyanine and aminolevulinic acid (ALA) in cultured cells have indicated that timing may be key factors in observation of any significant, effects, which may occur at longer temporal separations between radiation and PDT treatments (Ramakrishnan, N., et al 1990; Berg, K., et al. 1995). However, other studies have suggested that time sequences of the two treatments were not significant (Benstead, K., and Moore, J. V., 1990). In vivo studies have yielded mixed results, and to date, no study has specifically examined whether PDT can be delivered to target tissues in a manner that changes tissue metabolism such that, when used in conjunction with radiation therapy, a synergistic effect is seen.

Radiation therapy is the treatment of cancer and other diseases with ionizing radiation. Ionizing radiation deposits energy that injures or destroys cells in the area being treated (the “target tissue”) by damaging their genetic material, making it impossible for these cells to continue to grow. Although radiation damages both cancer cells and normal cells, the latter are able to repair themselves and function properly. Radiation may be used to treat localized solid tumors; it can also be used to treat leukemia and lymphoma (cancers of the blood-forming cells and lymphatic system, respectively).

One type of radiation therapy commonly used involves photons, “packets” of energy. X-rays were the first form of photon radiation to be used to treat cancer. Depending on the amount of energy they possess, the rays can be used to destroy cancer cells on the surface of or deeper in the body. The higher the energy of the x-ray beam, the deeper the x-rays can go into the target tissue. Linear accelerators and betatrons are machines that produce x-rays of increasingly greater energy. The use of machines to focus radiation (such as x-rays) on a cancer site is called external beam radiotherapy.

Gamma rays are another form of photons used in radiation therapy. Gamma rays are produced spontaneously as certain elements (such as radium, uranium, and cobalt 60) release radiation as they decompose, or decay. Each element decays at a specific rate and gives off energy in the form of gamma rays and other particles.

Several new approaches to radiation therapy are being evaluated to determine their effectiveness in treating cancer. One such technique is intraoperative irradiation, in which a large dose of external radiation is directed at the tumor and surrounding tissue during surgery.

Another investigational approach is particle beam radiation therapy. This type of therapy differs from photon radiotherapy in that it involves the use of fast-moving subatomic particles to treat localized cancers. A very sophisticated machine is needed to produce and accelerate the particles required for this procedure. Some particles (neutrons, pions, and heavy ions) deposit more energy along the path they take through tissue than do x-rays or gamma rays, thus causing more damage to the cells they hit. This type of radiation is often referred to as high linear energy transfer (high LET) radiation.

Methods for increasing the effectiveness of radiation therapy would be useful in the treatment of cancer.

### SUMMARY OF THE INVENTION

Photodynamic therapy depends on an oxygen-rich environment. Tumors can have large regions where oxygen is scarce, thereby limiting the effect of photodynamic therapy.

Radiation therapy is also known to be less effective in tumors with low oxygen, because the presence of oxygen increases the number of reactive species that induce toxic DNA strand breaks in tumor cells.

The present invention is based on the observation that photodynamic therapy can be conducted to halt cellular metabolism, thereby increasing available oxygen for redistribution to hypoxic areas. Thus, photodynamic therapy, when conducted to maximize oxygen production and distribution, and provided together with radiation therapy, can produce an enhanced therapeutic effect in the treatment of cancer.

In one aspect, the present invention provides a method of treating a tumor in a subject comprising administering a photosensitizer to the subject, irradiating the tumor between about 30 minutes to about 48 hours after administration of the photosensitizer such that cellular metabolism is decreased and oxygen is distributed to hypoxic areas of the tumor, and administering radiation therapy prior to, concurrently with or following photosensitizer administration and/or irradiation to thereby treat the tumor.

In one embodiment, blood flow within the tumor is preserved during treatment.

In another embodiment, the photodynamic therapy induces damage to the cellular fraction of the tumor, rather than to the blood vessels.

In a specific embodiment, the subject is a human.

In another specific embodiment, the tumor can be in the mouth, esophagus, stomach, small intestine, large intestine, trachea, larynx, lung, cervix, uterus, ovary, prostate, testicles or brain of the subject. The tumor can also be on the skin, or preferably not more than about 3 centimeters under the skin of the subject.

In another aspect, the present invention provides a method of decreasing malignant cell proliferation comprising contacting malignant cells with a photosensitizer that localizes to an intracellular compartment, irradiating the malignant cells such that cellular metabolism is decreased and oxygen is distributed to hypoxic cells, and providing radiation to the malignant cells prior to, concurrently with or following photosensitizer administration and/or irradiation to decrease proliferation of the malignant cells.

In one embodiment, the photosensitizer is cationic.

In a specific embodiment, the photosensitizer is Verteporfin or aminolevulinic acid (ALA), and is optionally coupled to a targeting moiety. In a specific embodiment, the targeting moiety is an antibody.

Irradiation is provided after an amount of time sufficient for intracellular localization of the photosensitizer. Preferably, the photosensitizer is localized in the mitochondrial membrane to decrease or inhibit intracellular metabolism. In a specific embodiment, irradiation is provided about 30 minutes to about 48 hours, more preferably about 1 to about 5 hours and even more preferably about 1 to about 3 hours following administration of the photosensitizer. In another specific embodiment, irradiation is provided about 3 hours after administration of the photosensitizer.

Irradiation can be applied directly to the tumor or malignant cell or by transillumination, and can be delivered, for example, with a laser or optical fiber.

Radiation therapy can be administered concurrently with or immediately after photodynamic therapy. In a specific embodiment, radiation therapy is administered in the form of x-rays.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

These and other objects and embodiments are described in or are obvious from and within the scope of the invention, from the following Detailed Description.

### BRIEF DESCRIPTION OF THE DRAWINGS

The following Detailed Description, given by way of example, but not intended to limit the invention to specific embodiments described, may be understood in conjunction with the accompanying Figures, incorporated herein by reference, in which:

FIGS. 1A-1D show a representative sample of antibodies having tumoricidal activity and/or tumor-specific epitope binding activity.

FIG. 2 shows tumor pO₂ measurements in RIF-1 tumors in mice treated with verteporfin-based photodynamic therapy and control animals with light alone but no photosensitizer (n=5). There were two treatment groups with each receiving 1 mg/kg of BPD in the verteporfin preparation (n=5 each). either 15 minutes or t 3 hours prior to light treatment. In both cases, the light was delivered with a total dose of 144 J/cm² at 690 nm wavelength. The error bars in the figure represent the standard deviation, and the values for the 3 hour treated group immediately after treatment (labeled ‘after Tx’ in graph) are significantly different from control values (p-value=0.048).

FIG. 3 shows cellular viability (closed symbols) and oxygen consumption rate (open symbols) as a function of the delivered light dose to RIF-1 cells incubated for 3 hours with 1 μg/ml BPD. Cell viability was determined using the MTS assay. Oxygen consumption rate was measured from cells in suspension using EPR oximetry. In both cases the data points represent the average of three successive trials, and the error bars are the standard deviation.

FIGS. 4A and 4B show calculated oxygen histograms from oxygen diffusion calculations where the metabolic oxygen consumption term (kₘₑₜ in equation 1) was varied. In FIG. 4A, kₘₑₜ=10 μM/s, whereas in FIG. 4B, kₘₑₜ was 0 μM/s. In both cases the simulations were carried out on the same capillary distribution patterns with D=2×10⁻⁵ cm²/s. In (a) the median pO₂ is 2 mm Hg and in (b) the median pO₂ is 7 mm Hg.

FIG. 5 shows average tumor volume plotted relative to the number of days after treatment (Days post treatment, on x-axis), for the five treatment groups; (i) control (n=6 mice), (ii) radiation only (n=5), (iii) photodynamic therapy alone (n=6); (iv) radiation followed by photodynamic therapy (n=7), and (v) photodynamic therapy together with radiation (n=7). The points represent the average values of each group in each group and the error bars are the standard error. The tumor volumes on the day of treatment and for doubling the treatment volume are denoted by horizontal dotted lines.

FIG. 6 shows oxygen measurements completed using EPR oximetry on groups of treated and control animals (n=5-6 each). The error bars show the average values during the first 2 minutes prior to light irradiation, average values during the entire treatment and then average values for the 2 minutes immediately following the light treatment. Oxygen values do not decrease at all during treatment, and oxygen concentrations rise significantly both during and after treatment for the conditions where low irradiance values were used.

FIGS. 7A and 7B show Eppendorf electrode measurements displayed as standard histograms of pO₂ (in units of mmHg) within the tumors measured, plotted as a function of the % frequency of each pO₂ value occurring. The data are pooled from three tracks in each animal with 7 control animals (FIG. 6A) and 6 treated animals (FIG. 6B). The treated animals received 100 mg/kg ALA injection 3 hours prior to irradiation with 150 mW/cm2 for 45 minutes. Control animals received the light alone without ALA, and the Eppendorf electrode measurements were taken from the tumors immediately after the light was turned off. The median pO₂ for the control group was 3.7 mmHg, and the median for the treated group was 8.7 mmHg.

### DETAILED DESCRIPTION

The present invention provides methods for the treatment of cancers (e.g., tumors, proliferating malignant cells) through the combined use of photodynamic therapy and radiation therapy. According to methods of the invention, photodynamic therapy is applied such that blood flow is preserved after treatment, and oxygen consumption by the tumor and/or surrounding parenchymal cells is reduced as a result of metabolic decline. As oxygenation of the tumor tissue increases, the effect of photodynamic therapy and radiation therapy also increases.

As used herein, the term “Photodynamic Therapy” or “PDT” comprises administration of a photosensitizer composition followed by irradiation thereof, such that a reactive species is produced. For purposes of this application, the method of treatment comprising photodynamic therapy and radiation can be referred to as “combination therapy.”

As used herein, the term “obtaining” as in “obtaining the photosensitizer” is intended to include purchasing, synthesizing or otherwise acquiring the photosensitizer.

I. Photosensitizers

As used herein, “photosensitizer” means a chemical compound that produces a biological effect upon photoactivation or a biological precursor of a compound that produces a biological effect upon photoactivation.

Photosensitizers known in the art can be selected for therapeutic uses according to: 1) efficacy in delivery, 2) proper localization in target tissues, 3) wavelengths of absorbance, 4) proper excitatory wavelength, and 5) purity and 6) in vivo effects on pharmacokinetics, metabolism, and reduced toxicity.

In specific embodiments, the photosensitizer selected for use in methods of the invention has a chemical structure that includes multiple conjugated rings that allow for light absorption and photoactivation, e.g., the photosensitizer can produce singlet oxygen upon absorption of electromagnetic irradiation at the proper energy level and wavelength.

Photosensitizers for clinical use are optimally amphiphilic, meaning that they advantageously share the opposing properties of being water-soluble, yet hydrophobic. A photosensitizer should be water-soluble in order to pass through the bloodstream systemically, however it should also be hydrophobic enough to pass across cell membranes. Modifications, such as attaching polar residues (amino acids, sugars, and nucleosides) to the hydrophobic porphyrin ring, can alter polarity and partition coefficients to desired levels. Photosensitizers having relatively high hydrophobicity, as measured, for example, by partition coefficient (e.g., a high partition coefficient indicates high hydrophobicity), are particularly suitable for methods of the present invention, due to facilitation of intracellular localization.

Photosensitizers of the present invention can bind to lipoproteins that are present in the bloodstream and be transported primarily to cells undergoing rapid division, such as tumors. Rapidly dividing cells require a greater amount of lipoproteins, and as a result, photosensitizers are selectively delivered to these cells at a higher level and with faster kinetics.

Preferably, photosensitizers of the present invention localize in the intracellular compartment, more preferably in the mitochondrial membrane. Both cationic and highly hydrophobic photosensitizers, for example, can localize in the mitochondrial membrane. Charge can be varied, for example, by conjugating charged residues, such as poly-L-lysine, to the photosensitizer.

Perferably, photosensitizers of the present invention absorb light at a relatively long wavelength, or in other words, can absorb at low energy. Low-energy light can travel further through tissue than high-energy light, which becomes scattered. In order to direct light to a region of interest, light scattering, i.e. the use of high-energy light, is not recommended, photodynamic therapy is predicated on the ability to produce a tumoridical effect in neoplastic cells by generation of singlet oxygen within them, and thus, it is necessary to use light that can travel through tissue. Optimal tissue penetration by light occurs between about 650 to about 800 nm. Accordingly, in order to absorb that light, a potential photosensitizer advantageously likewise absorbs low energy light. Porphyrins found in red blood cells typically absorb at about 630 nm, and new, modified porphyrins have optical spectra that have been “red-shifted”, in other words, absorbs lower energy light. Other naturally occurring compounds have optical spectra that is red-shifted with respect to porphyrin, such as chlorins found in chlorophyll (about 640 to about 670 nm) or bacteriochlorins found in photosynthetic bacteria (about 750 to about 820 nm).

Photosensitizers of the invention can be any known in the art, including the following:

A. Porphyrins

Porphyrins and synthetic, modified porphyrins have traditionally been used as photosensitizers in photodynamic therapy. Porphyrins are the backbones of the molecule heme, the chief constituent of hemoglobin, which is the carrier of oxygen in red blood cells. Porphyrins, in an oxygen-rich environment, can absorb energy from photons and transfer this energy to surrounding oxygen molecules. At a specific wavelength corresponding with that of incident light, porphyrin is excited to the singlet excited state (¹P*). This singlet excited porphyrin molecule can decay back to the ground state (P⁰) with release of energy in the form of fluorescence. If the lifetime of the singlet state is long enough, it is possible for the singlet state to be converted to a triplet excited state (³P*), which can transfer energy to another triplet state. A molecule that is present in great abundance in cells is oxygen, which naturally occurs in O₂ form. This dioxygen molecule has a triplet ground state, and provided that the energy of the ³P* molecule is higher than that of its product, dioxygen in its triplet state is converted into the highly toxic singlet oxygen.

As stated above, singlet oxygen, as well as free radicals that are also produced during the photoactivation process, is extremely reactive and can damage proteins, lipids, nucleic acids, and other cellular components. Cellular responses to singlet oxygen are complex, but in general, singlet oxygen causes phospholipid peroxidation leading to cell membrane damage and vessel occlusion-mediated ischemia, causing necrosis or apoptosis in the cell of interest. This mechanism of killing differs from cellular damage induced by radiation treatment, where γ-radiation is used to generate DNA double strand breaks which if unresolved, will ultimately result in cell death.

Porphyrins, hydroporphyrins, benzoporphyrins, and derivatives are all related in structure to hematoporphyrin, a molecule that is a biosynthetic precursor of heme, which is the primary constituent of hemoglobin, found in erythrocytes. First-generation and naturally occurring porphyrins are excited at about 630 nm and have an overall low fluorescent quantum yield and low efficiency in generating reactive oxygen species. Light at about 630 nm can only penetrate tissues to a depth of 3 mm, however there are derivatives that have been ‘red-shifted’ to absorb at longer wavelengths, such as the benzoporphyrins BPD-MA (Verteporfin). Thus, these ‘red-shifted’ derivatives show less collateral toxicity compared to first-generatio porphyrins.

Chlorins and bacteriochlorins are also porphyrin derivatives, however these have the unique property of hydrogenated exo-pyrrole double bonds on the porphyrin ring backbone, allowing for absorption at wavelengths greater than about 650 nm. Chlorins are derived from chlorophyll, and modified chlorins such as meta-tetra hydroxyphenylchlorin (mTHPC) have functional groups to increase solubility. Bacteriochlorins are derived from photosynthetic bacteria and are further red-shifted to about 740 nm.

Purpurins, porphycenes, and verdins are also porphyrin derivatives that have efficacies similar to or exceeding hematoporphyrin. Purpurins contain the basic porphyrin macrocycle, but are red-shifted to about 715 nm. Porphycenes have similar activation wavelengths to hematoporphyrin (about 635 nm), but have higher fluorescence quantum yields. Verdins contain a cyclohexanone ring fused to one of the pyrroles of the porphyrin ring. Phorbides and pheophorbides are derived from chlorophylls and have 20 times the effectiveness of hematoporphyrin. Texaphyrins are new metal-coordinating expanded porphyrins. The unique feature of texaphyrins is the presence of five, instead of four, coordinating nitrogens within the pyrrole rings. This allows for coordination of larger metal cations, such as trivalent lanthanides. Gadolinium and lutetium are used as the coordinating metals.

5-aminolevulinic acid (ALA) is a precursor in the heme biosynthetic pathway, and exogenous administration of this compound causes a shift in equilibrium of downstream reactions in the pathway. In other words, the formation of the immediate precursor to heme, protoporphyrin IX is dependent on the rate of 5-aminolevulinic acid synthesis, governed in a negative-feedback manner by concentration of free heme. Conversion of protoporphyrin IX is slow, and administration of exogenous ALA can bypass the negative-feedback mechanism and result in accumulation of phototoxic levels of ALA-induced protoporphyrin IX. ALA is rapidly cleared from the body, but like hematoporphyrin, has an absorption wavelength of about 630 nm, offering no advantage in terms of depth of tissue penetration.

Examples of porphyrin and porphyrin derivatives include but are not limited to Photofrin® RTM (porfimer sodium), hematoporphyrin IX, hematoporphyrin esters, dihematoporphyrin ester, synthetic diporphyrins, O-substituted tetraphenyl porphyrins (picket fence porphyrins), 3,1-meso tetrakis (o-propionamido phenyl) porphyrin, hydroporphyrins, benzoporphyrin derivatives, benzoporphyrin monoacid derivatives (BPD-MA), monoacid ring “a” derivatives, tetracyanoethylene adducts of benzoporphyrin, dimethyl acetylenedicarboxylate adducts of benzoporphyrin, endogenous metabolic precursors, δ-aminolevulinic acid, benzonaphthoporphyrazines, naturally occurring porphyrins, ALA-induced protoporphyrin IX, synthetic dichlorins, bacteriochlorins of the tetra(hydroxyphenyl) porphyrin series, purpurins, tin and zinc derivatives of octaethylpurpurin, etiopurpurin, tin-etio-purpurin, porphycenes, chlorins, chlorin e₆, mono-1-aspartyl derivative of chlorin e₆, di-1-aspartyl derivative of chlorin e₆, tin(IV) chlorin e₆, meta-tetrahydroxyphenylchlorin, chlorin e₆ monoethylendiamine monamide, verdins such as, but not limited to zinc methylpyroverdin (ZNMPV), copro II verdin trimethyl ester (CVTME) and deuteroverdin methyl ester (DVME), pheophorbide derivatives, and pyropheophorbide compounds, texaphyrins with or without substituted lanthanides or metals, lutetium (III) texaphyrin, gadolinium(III) and texaphyrin.

First-generation photosensitizers are exemplified by the porphyrin derivative Photofrin®, also known as porfimer sodium. Photofrin® is derived from hematoporphyrin-IX by acid treatment and has been approved by the Food and Drug Administration for use in photodynamic therapy. Photofrin® is characterized as a complex and inseparable mixture of monomers, dimers, and higher oligomers.

There has been substantial effort in the field to develop pure benzoporphyrin derivatives that can be used as photosensitizers. Thus, in one embodiment, the photosensitizer is a benzoporphyrin derivative (“BPD”), such as BPD-MA, also commercially known as Verteporfin®. U.S. Pat. No. 4,883,790 describes BPDs. Verteporfin® has been thoroughly characterized (Richter et al., 1987; Aveline et al., 1994; Levy, 1994) and it has been found to be a highly potent photosensitizer for photodynamic therapy. Verteporfin® is typically administered intravenously, with an optimal incubation time range from about 1.5 to about 6 hours. Verteporfin® absorbs at about 690 nM, and is activated with commonly available light sources.

Impairment of cellular metabolism has been demonstrated when Verteporfin is applied directly to cells both in vitro and in vivo (Pogue, B. W., et al. 2002). BPD-MA localizes to mitochondrial membranes, thereby specifically targeting the cellular organelle responsible for metabolism and respiration. Given that photosensitizers such as Verteporfin can localize intracellularly, (preferably within the mitochondrial membrane resulting in loss of mitochondrial membrane integrity), changes in light fluence, irradiance and localization of the photosensitizer can be modulated to alter cellular respiration, corresponding to transient flux in oxygen levels.

In another embodiment, a compound, e.g., ALA or ALA esters, which causes the accumulation of a photosensitizer, the formation of a photosensitizer, or is converted to a photosensitizer in the subject's body is administered to the subject. For example, a compound which causes the accumulation of, the formation of, or which is converted to, a porphyrin or a porphyrin precursor, is administered to the subject.

B. Photoactive Dyes

Cyanines are deep blue or purple compounds that are similar in structure to porphyrins. However, these dyes are much more stable to heat, light, and strong acids and bases than porphyrin molecules. Cyanines, phthalocyanines, and naphthalocyanines are chemically pure compounds that absorb light of longer wavelengths than hematoporphyrin derivatives with absorption maximum at about 680 run. Phthalocyanines, belonging to a new generation of substances for photodynamic therapy are chelated with a variety of metals, chiefly aluminum and zinc, while these diamagnetic metals enhance their phototoxicity. A ring substitution of the phthalocyanines with sulfonated groups will increase solubility and affect the cellular uptake. Less sulfonated compounds, which are more lipophilic, show the best membrane-penetrating properties and highest biological activity. The kinetics are much more rapid than those of HPD, with high tumor to tissue ratios (8:1) reached after about 1 to about 3 hours. The cyanines are eliminated rapidly and almost no fluorescence can be seen in the tumor after 24 hours.

Other photoactive dyes such as methylene blue and rose bengal, are also used for photodynamic therapy. Methylene blue is a phenothiazine cationic dye that is exemplified by its ability to specifically target mitochondrial membrane potential. Specific tumoricidal effects in response to cationic phenothiazine dyes are thought to be due to the electrical potential across mitochondrial membranes in tumor cells. Compared to normal cells, the potential in tumor cells is much steeper, leading to a high accumulation of compounds with delocalized positive charges (i.e. cationic photosensitizers). Rose-bengal and fluorescein are xanthene dyes that can be used in photodynamic therapy. Rose bengal diacetate is an efficient, cell-permeant generator of singlet oxygen. It is an iodinated xanthene derivative that has been chemically modified by the introduction of acetate groups. These modifications inactivate both its fluorescence and photosensitization properties, while increasing its ability to cross cell membranes. Once inside the cell, esterases remove the acetate groups and restore rose bengal to its native structure. This intracellular localization allows rose bengal diacetate to be a very effective photosensitizer.

Examples of photoactive dyes include but are not limited to Merocyanines, phthalocyanines with or without metal substituents, chloroaluminum phthalocyanine with or without varying substituents, sulfonated aluminum PC, ring-substituted cationic PC, sulfonated AlPc, disulfonated and tetrasulfonated derivative, sulfonated aluminum naphthalocyanines, naphthalocyanines with or without metal substituents and with or without varying substituents, tetracyanoethylene adducts, nile blue, crystal violet, azure β chloride, rose bengal, benzophenothiazinium compounds and phenothiazine derivatives including methylene blue.

C. Other Photosensitizers.

Other photosensitizers that do not fall in either of the aforementioned categories typically have other uses besides photodynamic therapy, but are also photoactive. For example, anthracenediones, anthrapyrazoles, aminoanthraquinone compounds are often used as anticancer therapies (i.e. mitoxantrone, doxorubicin). These drugs also have reasonable tumor selectivity. Chalcogenapyrylium dyes such as cationic selena- and tellurapyrylium derivatives have also been found to exhibit photoactive properties in the range of about 600 to about 900 nm (e.g., from about 775 to about 850 nm). In addition, antibiotics such as tetracyclines and fluoroquinolone compounds have demonstrated photoactive properties.

Examples of other photosensitizers include but are not limited to Diels-Alder adducts, dimethyl acetylene dicarboxylate adducts, anthracenediones, anthrapyrazoles, aminoanthraquinone, phenoxazine dyes, chalcogenapyrylium dyes such as cationic selena and tellurapyrylium derivatives, cationic imminium salts and tetracyclines.

D. Photoimmunoconjugates

Photosensitizers of the invention can optionally be linked to a targeting moiety that enhances intracellular localization. In a preferred embodiment, the targeting moiety is an antibody. The photosensitizer-antibody complex is called a photoimmunoconjugate (“PIC”). The photosensitizers can comprise a plurality of the same photosensitizer, each directly or indirectly linked to an antibody. In a preferred embodiment, the PIC comprises less than twenty of the same photosensitizer, each covalently linked to the antibody.

The use of PICs offers improved photosensitizer delivery specificity and could broaden the applicability of photodynamic therapy (photodynamic therapy). For example, it has been suggested that photodynamic therapy might be used effectively in the treatment of small diffuse malignancies present in a cavity, such as the peritoneum or bladder, if the photosensitizer could be made to accumulate with high specificity in malignant cells (Hamblin et al., 1996). This would allow photodynamic destruction of diseased cells while sparing adjacent normal tissues of sensitive organs. Many monoclonal antibodies known in the art possess tumoricidal activity. The combined therapeutic use of a tumoricidal antibody and a photosensitizer compound includes PICs wherein the monoclonal antibody component has an inhibitory effect on tumor growth. Tumoricidal antibodies, when used as monotherapy for reducing tumor growth, can have associated toxicity. Therapies requiring reduced levels of antibody administration can also reduce the occurrence of associated toxicity.

The antibody component of the PIC can bind with specificity to an epitope present on the surface of a tumor cell. “Binding with specificity” means that non-cancer cells are either not specifically bound by the antibody or are only poorly recognized by the antibody. The antibodies can comprise whole native antibodies, bispecific antibodies; chimeric antibodies; Fab, Fab′, single chain V region fragments (scFv) and fusion polypeptides. Preferably, the antibodies are monoclonal.

The term “antibody” as used in this invention includes intact immunoglobulin molecules as well as fragments thereof, such as Fab and Fab′, which are capable of binding the epitopic determinant. Fab fragments retain an entire light chain, as well as one-half of a heavy chain, with both chains covalently linked by the carboxy terminal disulfide bond. Fab fragments are monovalent with respect to the antigen-binding site.

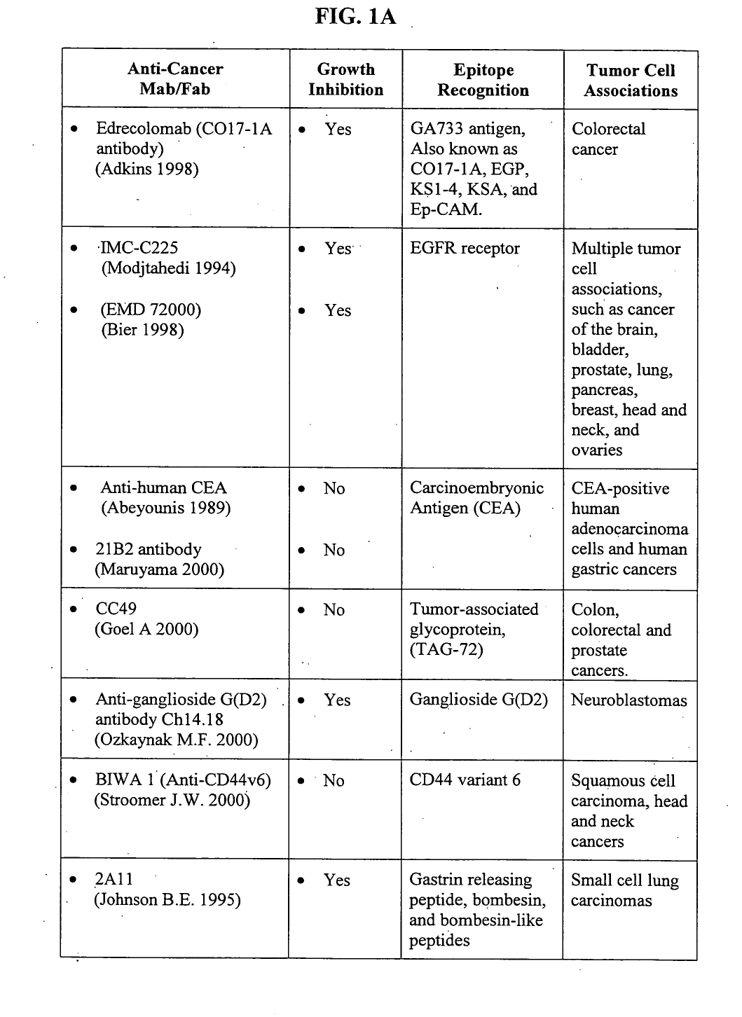

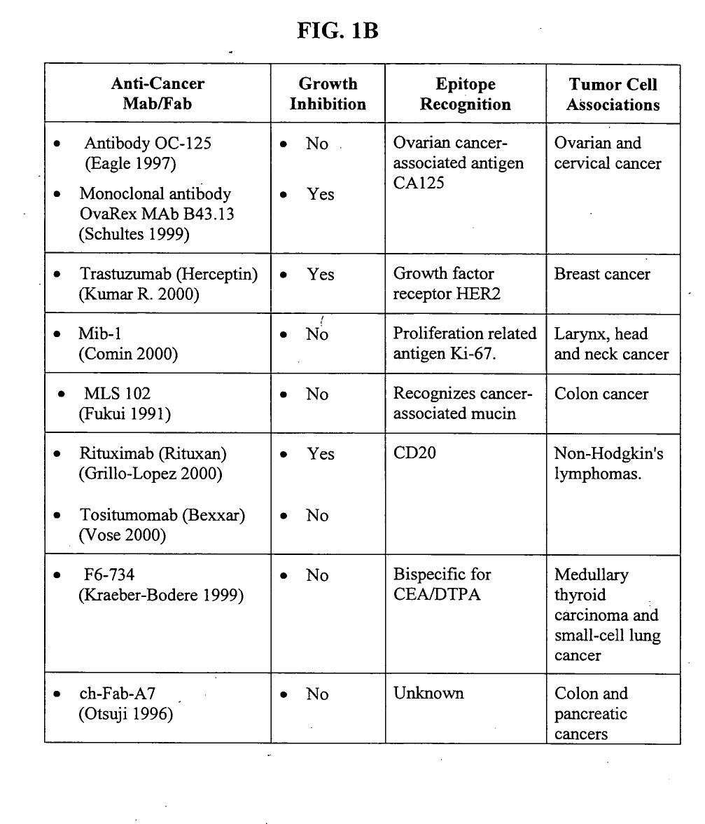

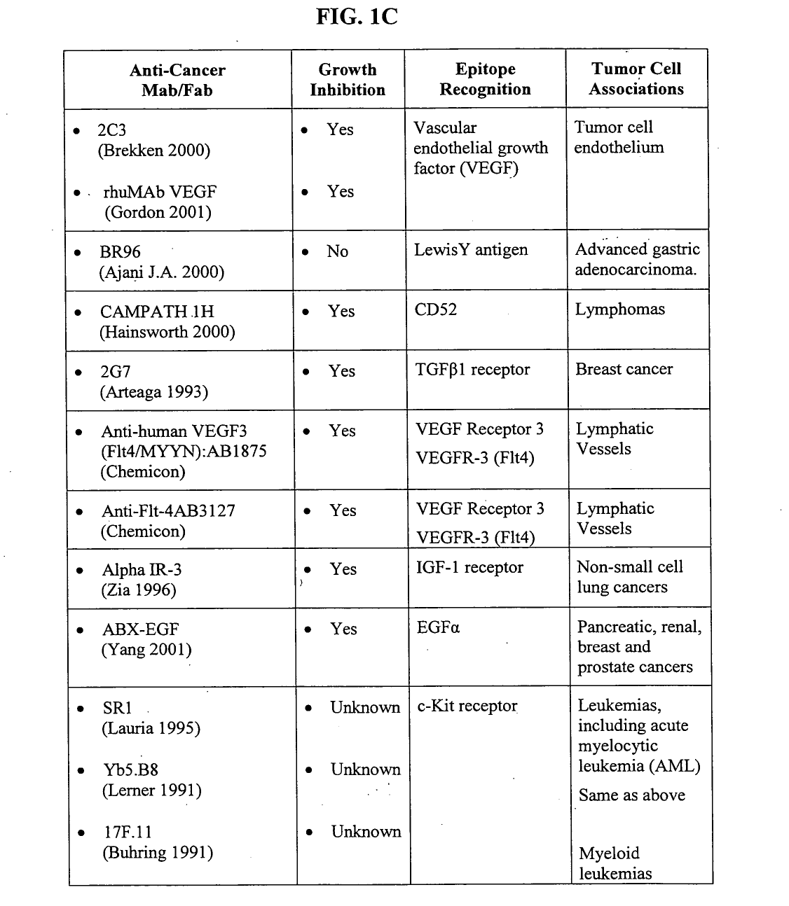

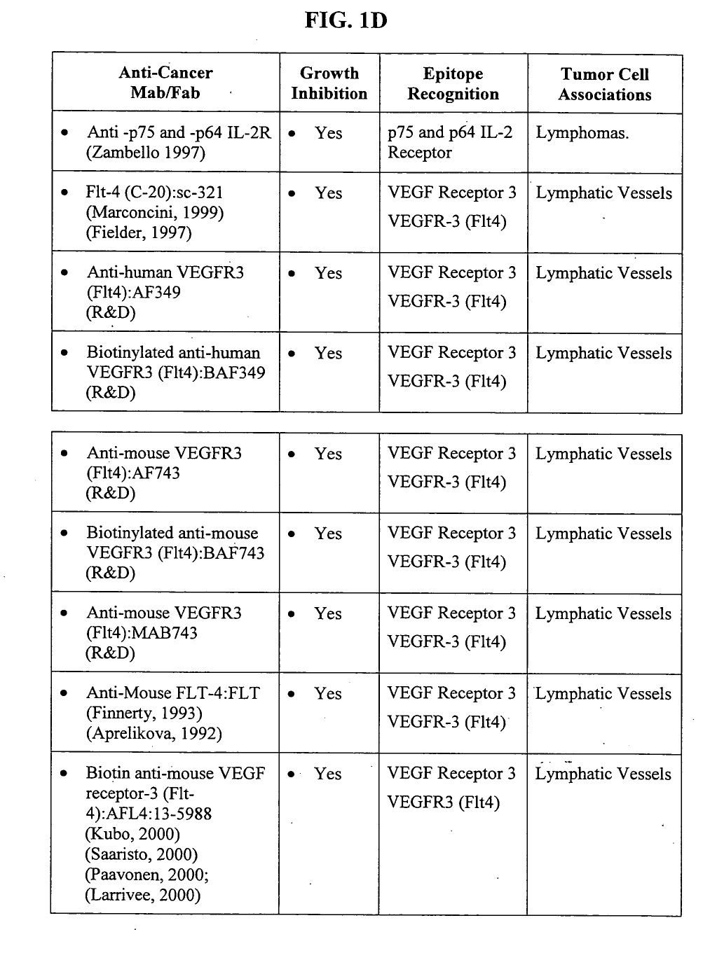

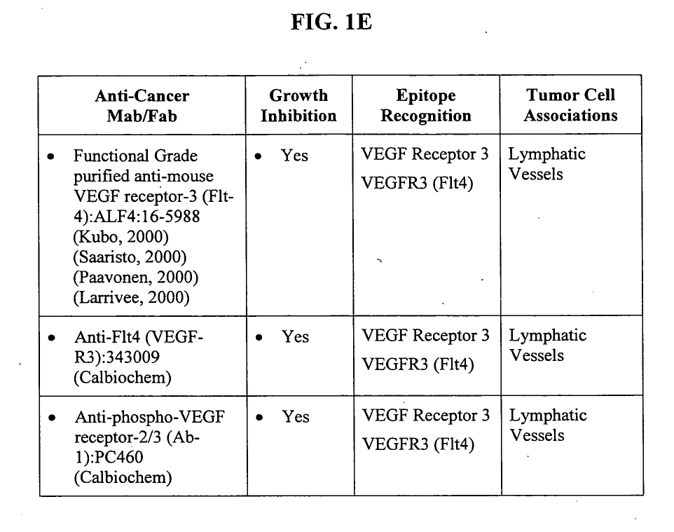

A representative sampling of tumor-specific antibodies is depicted in FIG. 1A-1D. For example, antibodies of the invention that bind to tumor cell epitopes include, but are not limited to, IMC-C225, EMD 72000, OvaRex Mab B43.13, 21B2 antibody, anti-human CEA, CC49, anti-ganglioside antibody G(D2) ch14.18, OC-125, F6-734, CO17-1A, ch-Fab-A7, BIWA 1, trastuzumab, rhuMAb VEGF, sc-321, AF349, BAF349, AF743, BAF743, MAB743, AB1875, Anti-Flt-4AB3127, FLT41-A, rituximab, tositumomab, Mib-1, 2C3, BR96, CAMPATH 1H, 2G7, 2A11, Alpha IR-3, ABX-EGF, MDX-447, SR1, Yb5.b8, 17F.11, anti-p75, anti-p64 IL-2R and MLS 102.

A wide variety of tumor-specific antibodies are known in the art, such as those described in U.S. Pat. Nos. 6,197,524, 6,191,255, 6,183,971, 6,162,606, 6,160,099, 6,143,873, 6,140,470, 6,139,869, 6,113,897, 6,106,833, 6,042,829, 6,042,828, 6,024,955, 6,020,153, 6,015,680, 5,990,297, 5,990,287, 5,972,628, 5,972,628, 5,959,084, 5,951,985, 5,939,532, 5,939,532, 5,939,277, 5,885,830, 5,874,255, 5,843,708, 5,837,845, 5,830,470, 5,792,616, 5,767,246, 5,747,048, 5,705,341, 5,690,935, 5,688,657, 5,688,505, 5,665,854, 5,656,444, 5,650,300, 5,643,740, 5,635,600, 5,589,573, 5,576,182, 5,552,526, 5,532,159, 5,525,337, 5,521,528, 5,519,120, 5,495,002, 5,474,755, 5,459,043, 5,427,917, 5,348,880, 5,344,919, 5,338,832, 5,298,393, 5,331,093, 5,244,801, and 5,169,774. See also The Monoclonal Antibody Index Volume 1: Cancer (3^{rd} edition). Accordingly, tumor-specific antibodies of the invention can recognize tumors derived from a wide variety of tissue types, including, but not limited to, breast, prostate, colon, lung, pharynx, thyroid, lymphoid, lymphatic, larynx, esophagus, oral mucosa, bladder, stomach, intestine, liver, pancreas, ovary, uterus, cervix, testes, dermis, bone, blood and brain.

As used in this invention, the term “epitope” means any antigenic determinant on an antigen to which the antibody binds. Epitopic determinants usually consist of chemically active surface groupings of molecules such as amino acids or sugar side chains and usually have specific three dimensional structural characteristics, as well as specific charge characteristics. Epitopes can be present, for example, on cell surface receptors.

Epitopes to which tumor-specific antibodies bind are also well known in the art. For example, epitopes bound by the tumor-specific antibodies of the invention include, but are not limited to, those known in the art to be present on CA-125, gangliosides G(D2), G(M2) and G(D3), CD20, CD52, CD33, Ep-CAM, CEA, bombesin-like peptides, PSA, HER2/neu, epidermal growth factor receptor, erbB2, erbB3, erbB4, CD44v6, Ki-67, cancer-associated mucin, VEGF, VEGFRs (e.g., VEGFR3), estrogen receptors, Lewis-Y antigen, TGFβ1, IGF-1 receptor, EGFα, c-Kit receptor, transferrin receptor, IL-2R and CO17-1A.

The antibodies can be prepared in several ways. Methods of producing and isolating whole native antibodies, bispecific antibodies, chimeric antibodies, Fab, Fab′, single chain V region fragments (scFv) and fusion polypeptides are known in the art. See, for example, Harlow and Lane (1988) Antibodies: A Laboratory Manual, Cold Spring Harbor Laboratory, New York (Harlow and Lane, 1988). Methods for conjugating photosensitizers and antibodies are described in Goff et al. (1991), Hamblin et al. (2001), Savellano et al. (2000), Jiang et al. (1990), Goers et al. (U.S. Pat. No. 4,867,973), and U.S. application Ser. No. 10/137,029, filed on May 1, 2002.

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology”, “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

In yet another embodiment, the antibody component of the PIC is a tumoricidal antibody. The term “tumoricidal antibody” as used herein refers to an antibody that inhibits tumor cell growth and/or proliferation through epitope binding. Antibodies that possess tumoricidal activity are also known in the art, including IMC-C225, EMD 72000, OvaRex Mab B43.13, anti-ganglioside G(D2) antibody ch14.18, CO17-1A, trastuzumab, rhuMAb VEGF, sc-321, AF349, BAF349, AF743, BAF743, MAB743, AB1875, Anti-Flt-4AB3127, FLT41-A, rituximab, 2C3, CAMPATH 1H, 2G7, Alpha IR-3, ABX-EGF, MDX-447, anti-p75 IL-2R, anti-p64 IL-2R, and 2A11.

In the context of the present invention, a tumor cell, which is also referred to herein as a malignant cell, is a cancer cell and a tumor is a mass of cancer cells, which can also encompass cells that support the growth and/or propagation of a cancer cell, such as vasculature and/or stroma. For instance, therefore, the present invention envisages compositions and methods for reducing growth of a tumor in a subject. In one embodiment, tumoricidial antibodies bind with specificity to cell surface epitopes (or epitopes of receptor-binding molecules) of a tumor cell or a cell that is involved in the growth and/or propagation of a tumor cell such as a cell comprising the vasculature of a tumor or blood vessels that supply tumors and/or stromal cells, such as parenchymal cells.

In a preferred embodiment, the antibody component of the PIC is IMC-C225, a chimeric therapeutic antibody made to the extracellular domain of the EGFR, which has shown great success in the treatment of head and neck cancer when administered in combination with radiation (Fan and Mendelsohn, 1998). Autocrine activation of the EGFR by EGF and TGF-α is important to tumor cell proliferation, and the EGFR appears to be an excellent target for anti-cancer therapies given that it is overexpressed in several types of tumors such as ovarian, colon, lung, and oral cancer (Perkins, 1997).

The lymphatic system is the primary pathway for the metastasis of most cancers. Activation of lymphatic endothelium by lymphangiogenic factors directly influences tumor progression by promoting tumor cell invasion and migration into the lymphatic vessels. VEGF-C and VEGF-D are members of the vascular endothelial growth factor (VEGF) family of angiogenic growth factors that have been identified as growth factors for lymphatic vessels. The induction of tumor lymphangiogenesis by VEGF-C results in increased infiltration of lymphatic vessels by tumor cells, and the extent of intratumoral lymphangiogenesis directly relates to the extent of tumor metastases. VEGFR-3, the receptor for VEGF-C and VEGF-D, is expressed in all tumor-associated lymphatic vessels and has been implicated in tumor lymphangiogenesis.

In a preferred embodiment, the antibody component of the PIC comprises an antibody to VEGFR-3. Tumoricidial antibodies to VEGFR-3 are known in the art. For example, sc-321 is commercially available from Bioscience (Santa Cruz, Calif.). Tumoricidial antibodies to VEGFR-3 include, but are not limited to AF349, BAF349, AF743, BAF743, MAB743, AB1875, Anti-Flt-4AB3127, and FLT41-A. PICs comprising tumoricidial antibodies to VEGFR-3 can be localized to the lymphatic vessels and selectively activated with light at the tumor site, causing local lymphatic vessel eradication.

In a preferred embodiment, the antibody component of the high-density PIC binds with specificity to a receptor or an epitope of a receptor-binding molecule present on the surface of a tumor cell. Antibodies of this category include, but are not limited to, IMC-C225, EMD 72000, BIWA 1, trastuzumab, rituximab, tositumomab, 2C3, rhuMAb VEGF, sc-321, AF349, BAF349, AF743, BAF743, MAB743, AB1875, Anti-Flt-4AB3127, FLT41-A, CAMPATH 1H, 2G7, alpha IR-3, ABX-EGF, MDX-447, SR1, Yb5.B8, 17F.11, anti-p75 IL-2R and anti-p64 IL-2R. Receptor epitopes or an epitope of a receptor-binding molecule include, but are not limited to those known in the art to be present on CD20, CD52, CD33, HER2/neu, epidermal growth factor receptor, erbB3, erbB4, CD44v6, VEGF, VEGFRs (e.g., VEGFR-3), estrogen receptors, TGFβ1, IGF-1 receptor, EGFα, c-Kit receptor, transferrin receptor, and IL-2R.

In yet another embodiment, binding of the antibody component of high-density PICs to the receptor epitope or an epitope of a receptor binding molecule inhibits growth and/or proliferation of the tumor cell. Tumoricidal antibodies in this category include, but are not limited to, IMC-C225, EMD 72000, trastuzumab, rituximab, 2C3, rhuMAb VEGF, sc-321, AF349, BAF349, AF743, BAF743, MAB743, AB1875, Anti-Flt-4AB3127, FLT41-A, CAMPATH 1H, 2G7, alpha IR-3, ABX-EGF, MDX-447, anti-p75 IL-2R and anti-p64 IL-2R.

The PICs can comprise, for example, at least one photosensitizer and at least one solubilizing agent each independently bound to an antibody through a direct covalent linkage, as described in U.S. application Ser. No. 10/094,120, published on Dec. 26, 2002 as US-2002-0197262-A1, the contents of which are incorporated herein by reference. The photosensitizer can be covalently bound, for example, through an amide linkage to a lysine residue of the antibody. The PICs of any one method can further comprise at least one photosensitizer covalently linked to an antibody, wherein the photosensitizer density on the antibody is sufficient to quench photoactivation while the composition is freely circulating throughout the bloodstream of a subject. Advantageously, these high-density PICs are dequenched following intracellular localization. Dequenching can occur, for example, by proteolytic, hydrolytic, or enzymatic intracellular processes, such as lysosomal degradation.

A wide variety of tumor-specific antibodies are known in the art. The antibody component of the PIC can bind with specificity to an epitope present on the surface of a tumor cell. Tumoricidal antibodies that bind with specificity to an epitope present on the surface of a tumor cell can be administered alone or in combination with a PIC also comprising a tumoricidal antibody recognizing a different epitope (the antibody and PIC should not compete with each other). The PIC composition administered to a subject can comprise a cocktail of tumor-specific antibodies, with or without tumoricidal activity, wherein the antibody component of the PICs, and optionally, the photosensitizer component, is variable from among the PIC of the composition. The cocktail would comprise only antibodies wherein epitope binding is non-competitive.

Photosensitizers of the present invention can optionally be linked to other targeting moieties known in the art, such as peptides that target cell surface receptors, preferably tumor cell surface receptors. Linkage can be achieved through the use of a coupling agent. The term “coupling agent” as used herein, refers to a reagent capable of coupling a photosensitizer to a targeting moiety, or to a “backbone” or “bridge” moiety. Any bond which is capable of linking the components such that they are stable under physiological conditions for the time needed for administration and treatment is suitable, but covalent linkages are preferred. The link between two components may be direct, e.g., where a photosensitizer is linked directly to a targeting moiety, or indirect, e.g., where a photosensitizer is linked to an intermediate, e.g., linked to a backbone, and that intermediate being linked to the targeting moiety. A coupling agent should function under conditions of temperature, pH, salt, solvent system, and other reactants that substantially retain the chemical stability of the photosensitizer, the backbone (if present), and the targeting moiety.

A coupling agent can link components without being added to the linked components. Other coupling agents result in the addition of elements of the coupling agent to the linked components. For example, coupling agents can be cross-linking agents that are homo- or hetero-bifunctional, and wherein one or more atomic components of the agent can be retained in the composition. A coupling agent that is not a cross-linking agent can be removed entirely during the coupling reaction, so that the molecular product can be composed entirely of the photosensitizer, the targeting moiety, and a backbone moiety (if present).

Many coupling agents react with an amine and a carboxylate, to form an amide, or an alcohol and a carboxylate to form an ester. Coupling agents are known in the art, see, e.g., M. Bodansky, “Principles of Peptide Synthesis”, 2nd ed., referenced herein, and T. Greene and P. Wuts, “Protective Groups in Organic Synthesis,” 2nd Ed, 1991, John Wiley, NY. Coupling agents should link component moieties stably, but such that there is only minimal or no denaturation or deactivation of the photosensitizer or the targeting moiety.

Photosensitizers of the invention can be prepared by coupling the photosensitizer to targeting moieties using methods known in the art. A variety of coupling agents, including cross-linking agents, can be used for covalent conjugation. Examples of cross-linking agents include N,N′-dicyclohexylcarbodiimide (DCC), N-succinimidyl-S-acetylthioacetate (SATA), N-succinimidyl-3-(2-pyridyldithio)propionate (SPDP), orthophenylenedimaleimide (o-PDM), and sulfosuccinimidyl 4-(N-maleimidomethyl) cyclohexane-1-carboxylate (sulfo-SMCC) (Karpovsky et al. (1984) J. Exp. Med. 160:1686; Liu, M A et al. (1985) Proc. Natl. Acad. Sci. USA 82:8648). Other methods include those described by Paulus and Behring (1985) Ins. Mitt., 78:118-132; Brennan et al. (1985) Science 229:81-83 and Glennie et al., (1987) J. Immunol,139:2367-2375. A large number of coupling agents for peptides and proteins, along with buffers, solvents, and methods of use, are described in the Pierce Chemical Co. catalog, pages T155-T-200, 1994 (3747 N. Meridian Rd., Rockford Ill., 61105, U.S.A.; Pierce Europe B.V., P.O. Box 1512, 3260 BA Oud Beijerland, The Netherlands), the contents of which are hereby incorporated by reference.

DCC is a useful coupling agent (Pierce #20320; Rockland, Ill.). It promotes coupling of the alcohol NHS to chlorin e6 in DMSO (Pierce #20684), forming an activated ester which can be cross-linked to polylysine. DCC(N,N′dicyclohexylcarbodiimide) is a carboxy-reactive cross-linker commonly used as a coupling agent in peptide synthesis, and has a molecular weight of 206.32. Another useful cross-linking agent is SPDP (Pierce #21557), a heterobifunctional cross-linker for use with primary amines and sulfhydryl groups. SPDP has a molecular weight of 312.4, a spacer arm length of 6.8 angstroms, is reactive to NHS-esters and pyridyldithio groups, and produces cleavable cross-linking such that, upon further reaction, the agent is eliminated so the photosensitizer can be linked directly to a backbone or targeting moiety. Other useful conjugating agents are SATA (Pierce #26102) for introduction of blocked SH groups for two-step cross-linking, which is deblocked with hydroxylamine-25-HCl (Pierce #26103), and sulfo-SMCC (Pierce #22322), reactive towards amines and sulfhydryls. Other cross-linking and coupling agents are also available from Pierce Chemical Co. (Rockford, Ill.). Additional compounds and processes, particularly those involving a Schiff base as an intermediate, for conjugation of proteins to other proteins or to other compositions, for example to reporter groups or to chelators for metal ion labeling of a protein, are disclosed in EPO 243,929 A2 (published Nov. 4, 1987).

Photosensitizers which contain carboxyl groups can be joined to lysine s-amino groups in the target polypeptides either by preformed reactive esters (such as N-hydroxy succinimide ester) or esters conjugated in situ by a carbodiimide-mediated reaction. The same applies to photosensitizers that contain sulfonic acid groups, which can be transformed to sulfonyl chlorides, which react with amino groups. Photosensitizers that have carboxyl groups can be joined to amino groups on the polypeptide by an in situ carbodiimide method. Photosensitizers can also be attached to hydroxyl groups, of serine or threonine residues or to sulfhydryl groups, of serine or threonine residues or to sulfhydryl groups of cysteine residues.

Methods of joining components of a composition, e.g., coupling polyamino acid chains bearing photosensitizers to polypeptides, can use heterobifunctional cross linking reagents. These agents bind a functional group in one chain and to a different functional group in the second chain. These functional groups typically are amino, carboxyl, sulfhydryl, and aldehyde. There are many permutations of appropriate moieties that will react with these groups and with differently formulated structures, to join them together (described in the Pierce Catalog and Merrifield et al. (1994) Ciba Found Symp. 186:5-20).

The production and purification of photosensitizers coupled to targeting moieties can be practiced by methods known in the art. Yield from coupling reactions can be assessed by spectroscopy of product eluting from a chromatographic fractionation in the final step of purification. The presence of uncoupled photosensitizer and reaction products containing the photosensitizer can be followed by the physical property that the photosensitizer moiety absorbs light at a characteristic wavelength and extinction coefficient, so incorporation into products can be monitored by absorbance at that wavelength or a similar wavelength. Coupling of one or more photosensitizer molecules to a targeting moiety or to a backbone shifts the peak of absorbance in the elution profile in fractions eluted using sizing gel chromatography, e.g., with the appropriate choice of Sephadex G50, 6100, or 6200 or other such matrices (Pharmacia-Biotech, Piscataway N.J.). Choice of appropriate sizing gel, for example Sephadex gel, can be determined by that gel in which the photosensitizer elutes in a fraction beyond the excluded volume of material too large to interact with the bead, i.e., the uncoupled starting photosensitizer composition interacts to some extent with the fractionation bead and is concomitantly retarded to some extent. The correct useful gel can be predicted from the molecular weight of the uncoupled photosensitizer. The successful reaction products of photosensitizer compositions coupled to additional moieties generally have characteristic higher molecular weights, causing them to interact with the chromatographic bead to a lesser extent, and thus appear in fractions eluting earlier than fractions containing the uncoupled photosensitizer substrate. Unreacted photosensitizer substrate generally appears in fractions characteristic of the starting material, and the yield from each reaction can thus be assessed both from size of the peak of larger molecular weight material, and the decrease in the peak of characteristic starting material. The area under the peak of the product fractions is converted to the size of the yield using the molar extinction coefficient.

The product can be analyzed using NMR, integrating areas of appropriate product peaks, to determine relative yields with different coupling agents. A red shift in absorption of a photosensitizer has often been observed following coupling to a polyamino acid. Coupling to a larger carrier such as a protein might produce a comparable shift, as coupling to an antibody resulted in a shift of about 3-5 nm in that direction compared to absorption of the free photosensitizer. Relevant absorption maxima and extinction coefficients in 0.1M NaOH/1% SDS are, for chlorinₑ₆, 400 nm and 150,000 M⁻¹, cm⁻¹, and for benzoporphyrin derivative, 430 nm and 61,000 M-1, cm^{-1.}

Photosensitizers compositions of the invention include those in which a photosensitizer is coupled directly to a targeting moiety, such as a scavenger receptor ligand. Other photosensitizer compositions of the invention include a “backbone” or “bridge” moiety, such as a polyamino acid, in which the backbone is coupled both to a photosensitizer and to a targeting moiety.

Inclusion of a backbone in a composition with a photosensitizer and a targeting moiety can provide a number of advantages, including the provision of greater stoichiometric ranges of photosensitizer and targeting moieties coupled per backbone. If the backbone possesses intrinsic affinity for a target organism, the affinity of the composition can be enhanced by coupling to the backbone. The specific range of organisms that can be targeted with one composition can be expanded by coupling two or more different targeting moieties to a single photosensitizer-backbone composition.

Peptides useful in the methods and compounds of the invention for design and characterization of backbone moieties include poly-amino acids which can be homo- and hetero-polymers of L-, D-, racemic DL- or mixed L- and D-amino acid composition, and which can be of defined or random mixed composition and sequence. These peptides can be modeled after particular natural peptides, and optimized by the technique of phage display and selection for enhanced binding to a chosen target, so that the selected peptide of highest affinity is characterized and then produced synthetically. Further modifications of functional groups can be introduced for purposes, for example, of increased solubility, decreased aggregation, and altered extent of hydrophobicity. Examples of nonpeptide backbones include nucleic acids and derivatives of nucleic acids such as DNA, RNA and peptide nucleic acids; polysaccharides and derivatives such as starch, pectin, chitins, celluloses and hemimethylated celluloses; lipids such as triglyceride derivatives and cerebrosides; synthetic polymers such as polyethylene glycols (PEGS) and PEG star polymers; dextran derivatives, polyvinyl alcohols, N-(2-hydroxypropyl)-methacrylamide copolymers, poly (DL-glycolic acid-lactic acid); and compositions containing elements of any of these classes of compounds.

The affinity of a photosensitizer composition can be refined by modifying the charge of a component of the composition. Conjugates such as poly-L-lysine chlorinₑ₆ can be made in varying sizes and charges (cationic, neutral, and anionic), for example, free NH2 groups of the polylysine are capped with acetyl, succinyl, or other R groups to alter the charge of the final composition. Net charge of a composition of the present invention can be determined by isoelectric focusing (IEF). This technique uses applied voltage to generate a pH gradient in a non-sieving acrylamide or agarose gel by the use of a system of ampholytes (synthetic buffering components). When charged polypeptides are applied to the gel they will migrate either to higher pH or to lower pH regions of the gel according to the position at which they become non-charged and hence unable to move further. This position can be determined by reference to the positions of a series of known IEF marker proteins.

II. Administration

A. Photosensitizer Administration

A “therapeutically effective amount” is an amount sufficient to effect a beneficial or desired clinical result. A therapeutically effective amount can be administered in one or more doses. In terms of treatment, an effective amount is an amount that is sufficient to palliate, ameliorate, stabilize, reverse or slow the progression of a cancerous disease (e.g. tumors, dysplaysias, leukemias) or otherwise reduce the pathological consequences of the cancer. A therapeutically effective amount is generally determined by the physician on a case-by-case basis and is within the skill of one in the art.

A therapeutically effective amount can be provided in one or a series of administrations. Photosensitizers of the invention can be administered topically or systemically. Standard texts, such as Remington: The Science and Practice of Pharmacy, 17th edition, Mack Publishing Company, incorporated herein by reference, can be consulted to prepare suitable compositions and formulations for administration, without undue experimentation. Suitable dosages can also be based upon the text and documents cited herein. A determination of the appropriate dosages is within the skill of one in the art given the parameters herein.

A “subject” is a vertebrate, preferably a mammal, more preferably a human. Mammals include, but are not limited to, humans, farm animals, sport animals, and pets. As a rule, the dosage for in vivo therapeutics or diagnostics will vary between subjects of the invention. Several factors are typically taken into account when determining an appropriate dosage. These factors include age, sex and weight of the subject, the condition being treated, the severity of the condition and the form of the antibody being administered.

In photodynamic therapy, dosage depends on various factors, including the amount of the photosensitizer administered, the wavelength of the photoactivating light, the intensity of the photoactivating light, and the duration of illumination by the photoactivating light. Thus, the dose of photodynamic therapy can be adjusted to a therapeutically effective dose by adjusting one or more of these factors.

The dosage of photosensitizer compositions can range from about 0.1 to about 10 mg/kg. Such dosages may vary, for example, depending on whether multiple administrations are given, tissue type and route of administration, the condition of the individual, the desired objective and other factors known to those of skill in the art.

The dosage of the PIC can vary from about 0.01 mg/m² to about 500 mg/m², preferably about 0.1 mg/m to about 200 mg/m², more preferably about 0.1 mg/m² to about 10 mg/m². Ascertaining dosage ranges is well within the skill of one in the art. For example, in phase three clinical studies, IMC-C225 loading in human patients was between 100-500 mg/m², and maintenance was between 100-250 mg/m² (Waksal, 1999).

Administrations can be conducted infrequently, or on a regular weekly basis until a desired, measurable parameter is detected, such as diminution of disease symptoms. Administration can then be diminished, such as to a biweekly or monthly basis, as appropriate.

Methods for administering photosensitizers are known in the art, and are described, for example, in U.S. Pat. Nos. 5,952,329, 5,807,881, 5,798,349, 5,776,966, 5,789,433, 5,736,563, 5,484,803 and by (Sperduto et al., 1991), (Walther et al., 1997). Photosensitizers are administered by a mode appropriate for the form of composition. Available routes of administration include subcutaneous, intramuscular, intraperitoneal, intradermal, oral, intranasal, intrapulmonary (i.e., by aerosol), intravenously, intramuscularly, subcutaneously, intracavity, intrathecally or transdermally, alone or in combination with tumoricidal antibodies. Photosensitizers are preferably administered intravenously. Therapeutic compositions of PICs are often administered by injection or by gradual perfusion.

Compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or liquid aerosol when used with an appropriate aerosolizer device. Although not required, compositions are preferably supplied in unit dosage form suitable for administration of a precise amount. Also contemplated by this invention are slow release or sustained release forms, whereby a relatively consistent level of the active compound are provided over an extended period.

Another method of administration is intralesionally, for instance by direct injection directly into the tumor. Intralesional administration of various forms of immunotherapy to cancer patients does not cause the toxicity seen with systemic administration of immunologic agents (Fletcher and Goldstein, 1987), (Rabinowich et al., 1987), (Rosenberg et al., 1986), (Pizza et al., 1984).

Methods of the invention are particularly suitable for use in treating and imaging brain cancer. When the site of delivery is the brain, the photosensitizer is advantageously capable of being delivered to the brain. The blood-brain barrier limits the uptake of many therapeutic agents into the brain and spinal cord from the general circulation. Molecules which cross the blood-brain barrier use two main mechanisms: free diffusion and facilitated transport. Because of the presence of the blood-brain barrier, attaining beneficial concentrations of a given therapeutic agent in the CNS may require the use of specific drug delivery strategies. Delivery of therapeutic agents to the CNS can be achieved by several methods.

One method relies on neurosurgical techniques. In the case of gravely ill patients, surgical intervention is warranted despite its attendant risks. For instance, therapeutic agents can be delivered by direct physical introduction into the CNS, such as intraventricular, intralesional, or intrathecal injection. Intraventricular injection can be facilitated by an intraventricular catheter, for example, attached to a reservoir, such as an Ommaya reservoir. Methods of introduction are also provided by rechargeable or biodegradable devices. Another approach is the disruption of the blood-brain barrier by substances which increase the permeability of the blood-brain barrier. Examples include intra-arterial infusion of poorly diffusible agents such as mannitol, pharmaceuticals which increase cerebrovascular permeability such as etoposide, or vasoactive agents such as leukotrienes (Neuwelt and Rapoport, 1984), (Baba et al., 1991), (Gennuso et al., 1993).

Further, it may be desirable to administer the photosensitizer locally to the area in need of treatment; this can be achieved, for example, by local infusion during surgery, by injection, by means of a catheter, or by means of an implant, said implant being of a porous, non-porous, or gelatinous material, including membranes, such as silastic membranes, or fibers. A suitable such membrane is Gliadel® provided by Guilford Pharmaceuticals Inc.

Methods of the invention are also particularly suitable for use in treatment of cancers of the mouth, esophagus, stomach, small intestine, large intestine, trachea, larynx, lung, cervix, uterus, prostate and testicles. In addition, combination therapy methods can be used to treat skin cancer, and tumors just below the surface of the skin. Laser light can pass through approximately 3 centimeters of tissue, so tumors on or beneath the surface of skin or on or near the lining of internal organs can be treated with minimal invasiveness.

Other potential uses include those where combination therapy could be combined with surgical debulking, such as pleural mesothelioma or advanced stage ovarian cancer. Currently, advanced ovarian cancer is treated by staging/debulking surgery, followed by chemotherapy, which is usually a combination of Taxol and platinum-based regimen. Rather than chemotherapy, combination therapy could instead be administered. For example, an administration scheme is envisioned whereby a photosensitizer is administered either before or after maximal debulking and subsequently photoactivated and subjected to x-ray radiation following the surgical procedure in order to eliminate residual cancer cells.

B. Administration of Photodynamic and Radiation Therapy

As used herein, “photoactivation” is interchangeable with “irradiation”, both of which mean a light-induced chemical reaction of a photosensitizer, which produces a biological effect.

In performing methods of the invention, irradiation is provided after an amount of time sufficient for intracellular localization of the photosensitizer, preferably in the mitochondrial membrane. In increase intracellular localization of the photosensitizer, one of skill in the art would readily vary parameters (e.g., the waiting period between administration of the photosensitizer and irradiation) depending of the characteristics of the photosensitized used. In a specific embodiment, irradiation is administered between 30 minutes and 48 hours, more preferably between about 1 to about 5 hours and even more preferably between about 1 to about 3 hours following administration of the photosensitizer. Duration of this “waiting step” varies, depending on factors such as route of administration, tumor location, and speed of photosensitizer movement in the body. In addition, where PICs target receptors or receptor binding epitopes, the rate of photosensitizer uptake can vary, depending on the level of receptor expression and/or receptor turnover on the tumor cells. For example, where there is a high level of receptor expression, the rate of PIC binding and uptake is increased. The waiting period should also take into account the rate at which PICs are degraded and thereby dequenched in the target tissue. Determining a useful range of waiting step duration is within ordinary skill in the art and may be optimized by utilizing fluorescence optical imaging techniques.

Following the waiting step, the photosensitizer is activated by photoactivating light applied to the tumor site or to malignant cells not associated with a tumor mass. This is accomplished by applying light of a suitable wavelength and intensity, for an effective length of time, specifically to the lesion site. The suitable wavelength, or range of wavelengths, will depend on the particular photosensitizer(s) used. Wavelength specificity for photoactivation depends on the molecular structure of the photosensitizer. Photoactivation occurs with sub-ablative light doses. Determination of suitable wavelength, light intensity, and duration of illumination is within ordinary skill in the art.

The light for photoactivation can be produced and delivered to the tumor site by any suitable means. For superficial tumors or open surgical sites, suitable light sources include broadband conventional light sources, broad arrays of light emitting diodes (LED), and defocussed laser beams.

For photoactivation, the wavelength of light is matched to the electronic absorption spectrum of the photosensitizer so that photons are absorbed by the photosensitizer and the desired photochemistry can occur. Target tissues can be illuminated, for example, with red light from a laser. Given that red and/or near infrared light best penetrates mammalian tissues, photosensitizers with strong absorbances in the approximately about 600 nm to about 900 nm range are optimal for photodynamic therapy. Delivery can be direct or by transillumination and can be delivered, for example, by laser or optical fiber (or both). Spatial control of illumination provides specificity of tissue destruction. In general, the amenability of lasers to fiberoptic coupling makes the task of light delivery to most anatomic sites manageable.

Light sources used in photodynamic therapy are typically lasers, which have the unique properties of being monochromatic, coherent (allows for precise focusing), and intense (shorter treatment times). Alternatives to lasers include the use of light emitting diodes (LEDs) and fluorescent light sources. Treatment times are advantageously increased if LEDs and fluorescent lights are used, as the light emitted from these sources are not as intense as lasers. The light is typically directed to the site of photosensitizer accumulation (target cells) through a fiber-optic, composed of a very thin strand of glass. The fiber-optic, when placed in close proximity to the cells of interest, delivers the proper amount of light. Fiber optics can be directed into place using various surgical equipment such as endoscopes for the esophageal tract, and bronchoscopes for lungs.

Optical fibers can be connected to flexible devices such as balloons equipped with light scattering medium. Flexible devices can include, for example, laproscopes, arthroscopes and endoscopes.

For non-superficial lesion sites, including those in intracavitary settings, the photoactivating light can be delivered by optical fiber devices. For example, the light can be delivered by optical fibers threaded through small gauge hypodermic needles. Optical fibers also can be passed through arthroscopes, endoscopes and laproscopes. In addition, light can be transmitted by percutaneous instrumentation using optical fibers or cannulated waveguides.

Photoactivation at non-superficial lesion sites also can be by transillumination. Some photosensitizers can be activated by near infrared light, which penetrates more deeply into biological tissue than other wavelengths. Thus, near infrared light is advantageous for transillumination. Transillumination can be performed using a variety of devices. The devices can utilize laser or non-laser sources, i.e. lightboxes or convergent light beams.

The effective penetration depth, δ_{eff}, of a given wavelength of light is a function of the optical properties of the tissue, such as absorption and scatter. The fluence (light dose) in a tissue is related to the depth, d, as: e^{-d}/δ_{eff}. Typically, the effective penetration depth is about 2 to about 3 mm at 630 nm and increases to about 5 to about 6 nm at longer wavelengths (e.g., 700-800 nm) (Svaasand and Ellingsen, 1983). These values can be altered by altering the biologic interactions and physical characteristics of the photosensitizer. Factors such as self-shielding and photobleaching (self-destruction of the photosensitizer during the photodynamic therapy) further complicate precise dosimetry. In general, photosensitizers with longer absorbing wavelengths and higher molar absorption coefficients at these wavelengths are more effective photodynamic agents.

As with photodynamic therapy, methods of administering radiation therapy are known in the art and are within the abilities of the skilled artisan to determine.

In a specific embodiment, administration of radiation therapy for treating a tumor in a subject occurs immediately following photosensitizer irradiation. “Immediately after” or “immediately following” means radiation is provided about 5 minutes to about 1 hour after the irradiation step of photodynamic therapy. Therapy provided in this manner has an unexpected synergistic effect on inhibition of tumor growth.

Typically, radiation therapy is administered either by external beam delivery from a linear accelerator or is applied interstitially through the use of implantible radioactive seeds, called brachytherapy. The standard for the majority of cancers is external beam irradiation where the delivery is given in fractionated doses, which are nominally 1 to 2 Gray of dose per day, given in one setting, and repeated on successive days for the total number of fractions to be achieved. Radiation can also be applied by Cobalt irradiators or orthovoltage irradiators when lower energy photons are desirable, such as in the case of superficial cancers. The choice of radiation given is decided upon by the accepted medical practice standard for each tumor type, and the choice of radiation delivery affects the penetration of the radiation as well as the dose pattern that is given to the tumor and normal surrounding tissues.

Radiation therapy is well known to be less effective in tumors with low oxygen partial pressure, because the presence of oxygen increases the number of reactive species generated by the radiation, and thereby increases the number of DNA strand breaks that occur. Methods to increase tumor tissue oxygenation have been shown to have a positive effect upon the ability to sensitize the tissue to radiation induced damage. Advantageously, methods of the present invention comprise increasing oxygen tension in the tissue together with cellular-targeting of photodynamic therapy, which can synergistically enhance radiation sensitivity in tumor tissue.

The present invention is additionally described by way of the following illustrative, non-limiting Examples, that provide a better understanding of the present invention and of its many advantages.

### EXAMPLES

### Example 1

### Measurement of Tumor Partial Pressure Oxygen in an In Vivo Model

Detection of oxygen partial pressure changes occurring in vivo during photodynamic therapy is difficult, as these changes are the result of many different factors (Veenhuizen, R. B., et al. 1995). Given the necessity of oxygen to be present for a tumoricidal effect, the consumption of oxygen in this process can be readily observed as an acute decrease of tissue partial pressure of oxygen (Tromberg, B. J., et al. 1990). Accordingly, it is thought that high optical dose rates could lead to less cell death due to the transient depletion of oxygen (Foster, T. H., et al. 1991). In addition to transient decreases in tumor oxygen, rapid and permanent reduction in blood flow and tumor oxygenation can occur due to vascular occlusion during or soon after photodynamic therapy when a large amount of excess photosensitizer is present in blood vessels (Iinuma, S., et al. 1999). This is the theory behind the use of photodynamic therapy in macular degeneration, where unwanted growth of blood vessels of the retina is inhibited. Iinuma and coworkers demonstrated that Verteporfin is confined to the vasculature after 5 minutes of photosensitizer injection, but diffuses to other regions after 1 hour. Additionally, studies have shown that blood vessels dilate after Verteporfin treatment, transiently increasing blood flow after photodynamic therapy. Taken together, when irradiance is applied hours after initial photosensitizer injection, the blood vessel does not occlude, and blood flow may be preserved during photodynamic therapy therapy.

RIF-1 Cell Culture and Injection into Mice

Measurements of tumor partial pressure of oxygen (pO₂) were taken in mice that harbored tumors derived from injection of transformed cells. Radiation-induced fibrosarcoma (RIF-1) cells were used for in vivo studies. RIF-1 cells were obtained from the National Cancer Institute (NCI) and originated from the laboratory of James B. Mitchell. The cells were grown in culture in RPMI 1640 cell culture medium, supplemented with 10% fetal bovine serum (FBS), L-glutamine, and appropriate antibiotics. The cells were not passaged more than four times from the original stock. Cell cultures were scaled up to large culture flasks, then removed and resuspended in medium lacking FBS and injected at 4×10⁶ cells/mouse in a 50 μl volume. Intradermal injection delivered the cancer cells into the upper right leg of female C3h/HeJ mice (age 5-6 weeks) approximately 11-14 days before the anticipated treatment time.

Preparation and Delivery of Photosensitizer Agent

Verteporfin was obtained from QLT Phototherapeutics Inc. (Vancouver, BC, Canada) for use as photosensitizer. The photoactive molecule is a benzoporphyrin derivative mono-acid ring ‘A’ (BPD-MA), which was suspended in a lipid preparation comprising 1.77% of BPD-MA and the remaining 98.23% composed of the lipids. Verteporfin was reconstituted in phosphate buffered saline (PBS) at a final concentration of 0.2 mg BPD-MA/ml, then injected into mice lateral tail vein to deliver a dose of 1 mg BPD-MA per kg of body weight. The drug was allowed to incubate for varying times prior to light exposure.

Light Delivery

A diode laser system with 200 mW average power was used, at a wavelength of 690 nm (Applied Optronics, South Plainfield, Conn.). The beam was delivered to the tumor site in animals through a 140 μm fiber-optic, and was expanded onto the tumor in a circular top-hat beam, using a fiber-optic collimator (Thor Labs, North Newton, N.J.). The beam diameter for in vivo experiments was 1.1 cm, producing an irradiance of 200 mW/cm². Light treatment was given transcutaneously with shaven animals.

Cellular pO₂ Measurement

Animals were anesthetized by isoflurane at a concentration of 1.5% mixed with 26% oxygen in a continuous flow, delivered by inhalation. The animal was maintained at 37° C. on a heated water pad. Warm air was allowed to flow over the animal. In the first treatment group, tumors were exposed to light three hours after verteporfin injection. The second group received light only 15 minutes after injection. These animals were monitored for tumor pO₂ before, during, and after treatment. The second experimental group is thought to be indicative of eliciting a vascular occlusion response, and contrasts effects from 3-hour photosensitizer incubation. The third group received light without prior photosensitizer injection.

Tumor pO₂ was monitored with an oxygen-sensitive electron paramagnetic resonance (EPR) probe material, synthetic lithium phthalocyanine (LiPc), which was implanted into tissue and allowed for stable measurements of tissue pO₂ over several weeks. Initial experiments performed revealed that in the absence of interactions between the probe and the photosensitizer or light, pO₂ could be monitored with EPR during treatment. LiPc molecules between 50 and 200 μm in diameter were implanted into the animal tumors with a 23-gauge needle at least 24 hours before photodynamic therapy, allowing for resolution of acute fluctuations in pO₂ due to injection. The particles were implanted at a depth of 1-3 mm within the tumor.

The animals were placed in an L-band (1.2 GHz) EPR spectrometer with a custom-made microwave bridge for measurements of tissue pO₂. The external loop resonator was positioned over the tumor to record the EPR signal of the LiPc from within the tissue. Typical settings for the spectrometer were: incident microwave power of 50 mW; magnetic field of 400 Gauss, scan range of 0.5 Gauss; modulation frequency of 27 kHz; modulation amplitude of 15 mGauss; scan time of 30 seconds. The settings remained similar throughout the experiments, and between animals. After accumulation of linewidths as a function of time before, during, and after treatment, the data were fit with a custom-written software program to match the Voigt line shape of the EPR spectrum and extract the line width. LiPc was pre-calibrated to determine the ambient pO₂ from the line width, allowing calculation of effective tissue pO₂ during the experiment.

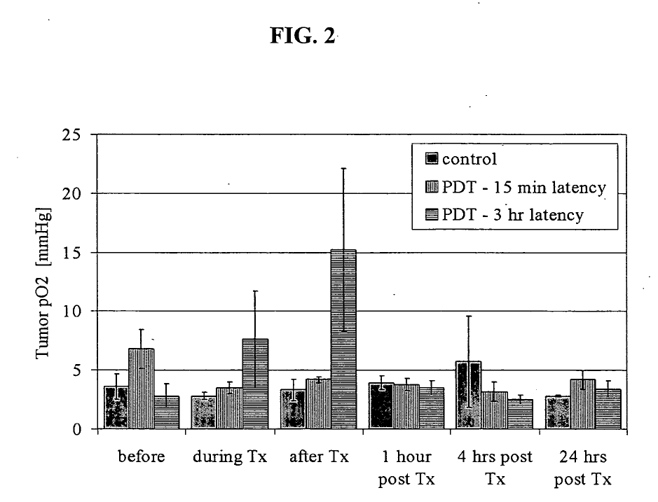

Measurements of tumor pO₂ were taken in control and verteporfin-injected tumor-bearing animals. Mean (±SD) tumor volume for all groups at the time of treatment was between 203 mm³±23 mm³ (range 110-300). At the time of treatment, there were no significant differences between the tumor volumes of the groups. The EPR probe was placed within the top 2-3 mm of tissue to ensure that it was at the site of the tumor. This measurement was repeated in each animal, at several different time-points before, during, and after treatment. The mice were re-anesthetized at 1 h, 4 h, and 24 h post-treatment for pO₂ measurement. The average values at each time point were calculated for each animal separately (before, during, and after treatment at 1 h, 4 h, and 24 h). The pO₂ values for each group of animals were averaged and standard deviations calculated (FIG. 2).

Control animals were irradiated with laser light, but were given no photosensitizer injection. The control animals all had initial pO₂ values of 3.6±1.1 mm Hg, indicating that the tumors have a high hypoxic fraction. Throughout and after laser treatments, the control animals maintained similar hypoxic pO₂ values. The treatment group displayed initial pO₂ values of 2.8±1.0 mm Hg. Initial changes in pO₂ varied between animals, however, by the end of the treatment period, the pO₂ of all five of the treated tumors had risen significantly. The final pO₂ value obtained immediately after treatment was 15.2±6.9 mm Hg, markedly different from the control value. The difference was calculated by a paired students-t test with a p-value of 0.048. The pO₂ values returned to within control, or pre-treatment values at 1 h post-treatment.

The second group of animals, which were injected just 15 min prior to irradiation, displayed average tumor pO₂ values of 6.8±1.6 mm Hg prior to light exposure, and 4.1±0.3 mm Hg immediately following treatment. This second group represents “vascular targeting” type of therapy and tested the efficacy of photodynamic therapy without the long photosensitizer incubation.

### Example 2

### In Vitro Cellular Oxygen Consumption and Viability.

Preparation of RIF-1 Cells for In Vitro Photodynamic Therapy

RIF-1 cells were plated three days prior to treatment in black, plastic 96-well plates with a transparent bottom (Fisher Scientific, Springfield, N.J.) at a density of 5000 cells/200 μl medium/well. After 3 days of growth in 5% CO₂ at 37° C. in a humidified incubator, the cells were used in photodynamic therapy. On the day of treatment, the medium was replaced with 100 μl/well of medium with 1 μg/ml BPD in the verteporfin preparation. After a 3-hour incubation, the BPD solution was removed and rinsed once with Hanks Balanced Salt Solution (HBSS), then 100 μl of fresh medium was added to the wells. Cells were irradiated in groups of four wells, with blank wells between the treated groups to ensure that each group of wells received the correct dose of light. In each 96-well plate, squares of 4 were treated with increasing light doses with 8 groups/plate, including the control, non-irradiated group. Surviving cells were assayed for viability and/or cellular oxygen consumption.

Cell Viability Assay

Surviving cells were assayed 24 hours after treatment, using the MTS assay kit (CellTiter 96®AQueous, Promega, Madison, Wis.). This assay is composed of [3-(4,5-dimethylthiazol-2-yl)-5-(3-carboxymethoxyphenyl)-2-(4-sulfophenyl)-2H— tetrazolium, inner salt; MTS^{(a)}] and an electron coupling reagent (phenazine methosulfate) PMS. MTS becomes reduced by cellular mitochondrial dehydrogenases into a soluble, optically active formazan product. After photodynamic therapy, the medium from the 96-well plate was removed and the MTS tetrazolium reagent added. After incubation for 1 h at 37° C., the absorbance of the formazan product at 490 nM was measured directly in an automated plate reader (ThermoMax, Molecular Devices, Menlo Park, Calif.). The quantity of formazan product as measured by the amount of 490 nm absorbance units is directly proportional to the number of living cells in culture, and absorbance values from groups of wells that received the same light dose were averaged. The data were normalized by the average value from the control wells, which received no light.

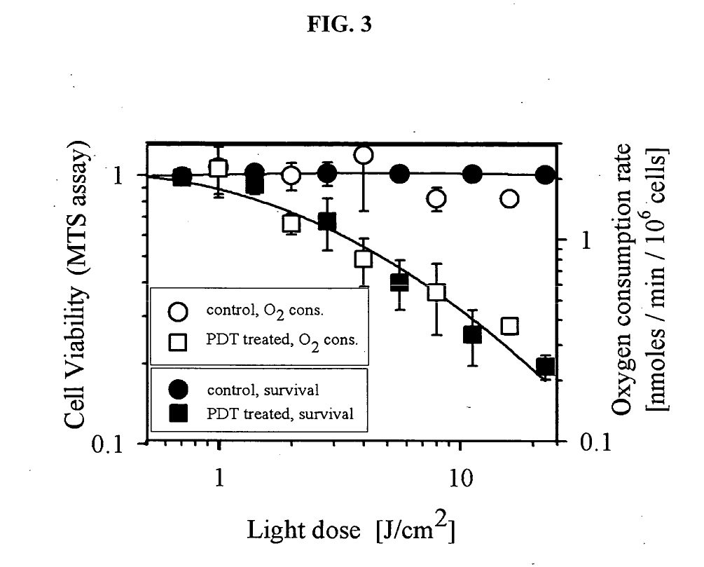

The cell viability assay data is shown in FIG. 3, represented as closed symbols, with survival plotted on the left-hand vertical axis. Data were normalized to 1.0 for untreated cells. The cell viability assay was plotted on the same graph as the data obtained from the cellular oxygen consumption assays to demonstrate the similarity between the dose responses.

In Vitro Cellular pO₂ Measurement

Immediately following photodynamic therapy, cells were trypsinized, suspended in fresh medium, and cell viability determined by trypan blue dye exclusion prior to use in oxygen consumption measurement. Cells were resuspended at 2×10⁷ viable cells/ml in complete medium containing 10% dextran. The rate of oxygen consumption was measured using EPR. Cell suspensions containing dextran were extracted in 100 μl volumes and mixed with neutral nitroxide, ¹⁵N photodynamic therapy (4-oxo-2,2,6,6-tetramethylpiperidine, d₁₆-¹⁵N-1-oxyl) at 0.2 mM (Cambridge Isotope Laboratories, Quebec, Canada) in a 4 μl volume. This mixture was removed with glass capillary tubes and then sealed with Critoseal (Sherwood Medical, St. Louis, Mo.). The sealed tubes were placed into quartz ESR tubes and samples were kept at 37° C. by a heated flow of gas through the resonator. A Bruker EMX EPR spectrometer measured all spectra, operating at 9 GHz. Since the resulting linewidth was proportional to oxygen concentration, oxygen consumption rates were obtained by measuring the concentration repeatedly over 10-20 min. The slope of the obtained data was determined by linear regression. Three repeated trials were performed for each sample of cells, and the slope values were averaged.

The oxygen consumption rates of untreated cells were higher than those obtained from cells treated with BPD-PDT (FIG. 3). The average oxygen consumption rate in the untreated cells was measured at 1.84±0.14 nmoles per minute per million cells. When treated with light alone, small increases in the oxygen consumption rate were observed on the order of ±0.2 mmol/minute/10⁶ cells. When treated with photosensitizer and light, there was a monotonic decrease in oxygen consumption that correlated to the delivered light dose. At the highest light dose of 16 J/cm², the oxygen consumption rate dropped to 0.37±0.04 mmol/min/10⁶ cells. The data for these measurements are shown in FIG. 3 (open symbols; treated cells are represented by squares; control cells are represented by circles). Absolute units of oxygen consumption are plotted on the right-hand vertical axis. Control groups received light doses in the absence of photosensitizer. Examination of the dose responses between the oxygen consumption assays and the cell viability assays revealed that they are quantitatively identical. At the maximal light dose, oxygen consumption is 21±4% of control oxygen consumption rates, and accordingly, the cell viability assay showed a decrease to 20±5% cell survival.

### Example 3

### Changes in pO₂ Based on Changes in Metabolic Consumption

Oxygen Distribution in RIF-1 Tumors

A finite element solution to the steady state diffusion equation was applied to solve for the oxygen concentration within an arbitrary volume of tissue. The differential diffusion equation is represented for steady state levels:
D∇²C_{O2}(r)−kₘₑₜ(r, O₂)+S_{O2}(r)=0
Where Cₒ₂(r) is the oxygen concentration at position r, D is the diffusion coefficient for oxygen in tissue (spatially independent), kₘₑₜ(r,O₂) is the metabolic oxygen consumption rate, and S_{O2}(r) is the supply of oxygen by the capillaries at each point r.

The geometries of the capillary tubes containing the tissue samples were derived from eight H&E stained sections of RIF-1 tumor tissue. These were digitized and manually thresholded. The capillary oxygen supply rates were estimated based upon fitting to boundary information, given by pimonidazole staining of adjacent sections of the tissue. Pimonidazole staining yields demarcation of regions of hypoxia, thus allowing for estimation of the neighboring capillary oxygen supply rates, assuming that the oxygen diffusion coefficient was known (D=2×10⁻⁵ cm²/s).

The eight sections of tumor tissue were used to simulate oxygen distribution within the tumor. The oxygen supply rates of the capillaries were shown by the previous pimonidazole data fitting. The value of kₘₑₜ was varied to demonstrate changes anticipated in the tumor under normal conditions (i.e. kₘₑₜ=10 μM/s), and under conditions where metabolic consumption is stopped (i.e. kₘₑₜ≅0 μM/s). A histogram was generated, demonstrating the calculated oxygen distributions in a format typically obtained with an Eppendorf electrode. All calculations were completed in concentration units of μM, then converted to units of pO₂ (mm Hg), by multiplication by the solubility of oxygen in water, assuming that the solubility of the tumor tissue is similar.

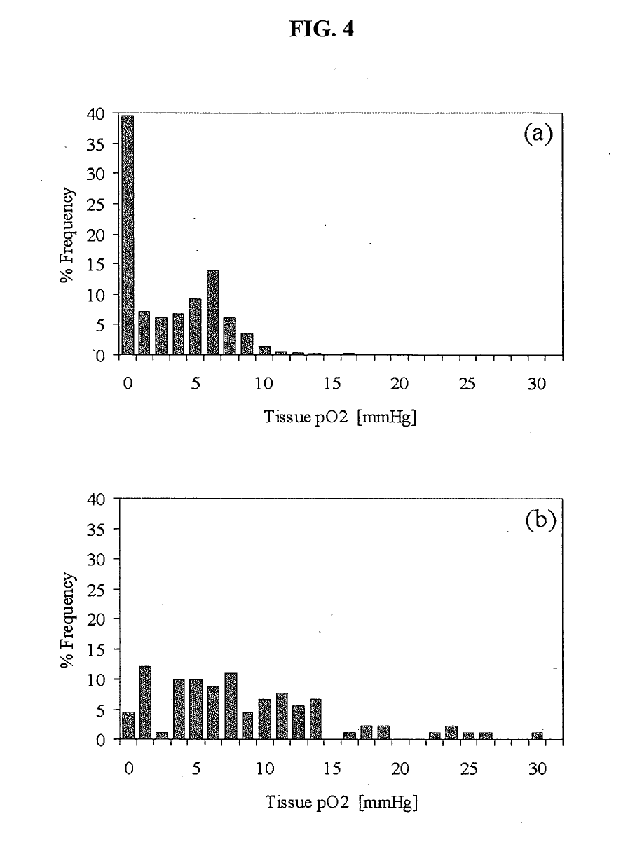

Oxygen histograms were simulated for the two conditions of kₘₑₜ=10 μM/s and kₘₑₜ=0 μM/s. The values of pO₂ were calculated from the images used, and the data was presented in FIGS. 4A and 4B, in cumulative format. Median pO₂ values for the two distributions were 2 mm Hg and 7 mm Hg, respectively. This indicates that when metabolic oxygen consumption rates are minimal, the pO₂ of the tumor tissue could increase by a median of 5 mm Hg. The data predict that the hypoxic fraction (the fraction of the tumor less than 10 mm Hg pO₂) is initially 98%, whereas after changes in metabolic consumption, the hypoxic fraction is reduced to 65%.

### Example 4

### Combined Photodynamic Therapy and Radiation Therapy

The combination effect of photodynamic therapy and radiation therapy was examined in in vivo tumor models. Injection of PBD or saline (control) was given 3 hours prior to irradiation in all animals. Light at 690 nm wavelength was delivered t the photodynamic therapy treated animals while the animal was positioned in the irradiator cone. An irradiance of 133 mW/cm² was used for a total of 12 minutes, for a total dose of 100 J/cm². Radiation was administered at a single dose of 10 Gy (300 keV, 10 mA, HVL=2.33 Gy/min). The following groups were organized: (1) sham-irradiated controls, (2) radiation treatment alone, (3) photodynamic therapy alone, (4) radiation treatment followed by photodynamic therapy, and (5) photodynamic therapy with simultaneous radiation treatment. In Group 4, X-ray radiation was administered for the full 3-minute treatment period after the photosensitizer was injected and incubated in the animal for 3 hours, then irradiation was given for 12 minutes. The Group 5 animals received irradiation 3 hours after drug injection, and during the last three minutes of the irradiation. X-ray irradiation was also given to the tumor at the same time. The latter timing resulted in the tumor's exposure to X-rays during the maximal re-oxygenation time of the tumor tissue.

Irradiation using the 10 Gy dose using approximately 300 keV was delivered in 3 minutes from a Pantak Therapax 300 orthovoltage irradiator, using a 2 cm collimation cone. This cone was contacted with a region of the mouse leg surrounding the tumor. The end of the collimation cone was comprised of transparent plastic, allowing radiation and treatment to occur at the same time (required for Group 5, where photodynamic therapy and radiation were simultaneously administered). All animals were treated with the cone in place, as well as Group 1 sham-irradiated mice and Group 3 photodynamic therapy alone mice. This was performed to ensure equal doses of light were given to all groups, which included changes in the beam from reflection and refraction through the irradiator cone.

Tumor Regrowth Assay

Each group of mice were assayed for tumor regrowth following combined radiation/photodynamic therapy, radiation alone, or photodynamic therapy alone. The resulting treatment effect was assayed by calculation of tumor volume, as calculated by measurement of the three major axis dimensions:
Volume≅Length×Width×Height/2
The time for a tumor to reach double its volume on the day of treatment was calculated for each individual animal separately, by estimating the data by a linear fit to logarithmic growth curves versus time. The average values from these times to double in volume were determined for each group of mice. Analysis of the tumor regrowth data, estimates of the mean double time (including standard deviation), and mice heterogeneity was achieved by generating individual growth curve data and fitting with a mixed effect model. It was assumed that after day 3, the tumor re-grows exponentially, which corresponds to a linear function when plotted on a semi-logarithmic graph. The time for a tumor to reach double its volume on the day of treatment was calculated for each group along with the respective standard deviation. The standard error gives rise to the group comparison using X-test to account for significant differences between the mean values of the different treatment groups.

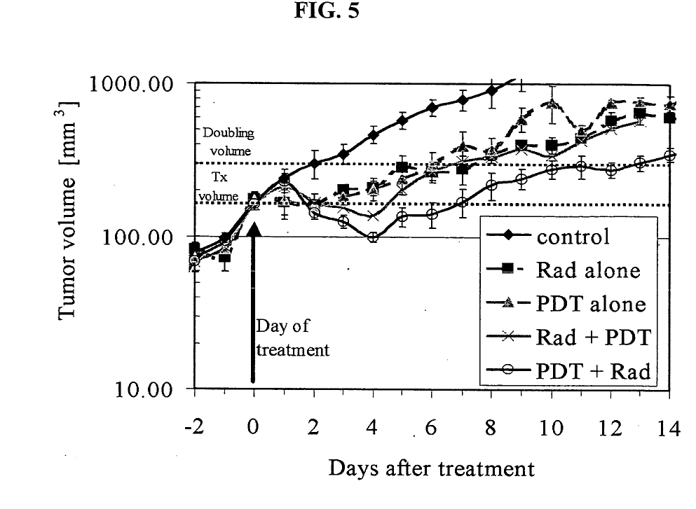

FIG. 5 depicts the measurements of tumor volumes after treatment with radiation, photodynamic therapy alone, or combined photodynamic therapy and radiation. The graph in FIG. 5 shows that tumor regrowth rate in all groups is slightly lower than that of the control group, but the slopes are similar between all treated groups. The slopes of the natural logarithm of tumor volume versus days were, for each treatment group: 0.20 (Group 1); 0.11 (Group 2); 0.13 (Group 3); 0.13 (Group 4); 0.11 (Group 5). Group 3 (photodynamic therapy alone) and Group 2 (radiation alone) induce the same approximate time for the tumor to reach twice the volume of the treatment size. In comparison, group 4 (radiation treatment followed by photodynamic therapy), and Group 5 (photodynamic therapy with simultaneous radiation treatment) demonstrated increasingly better effects. The numbers are included in Table 1, while the p-values comparing various groups are shown in Table 2.

TABLE 1
Tumor Doubling Times and Re-Growth Delay

| | | Time for Tumor to | Re-growth Delay |
|---|---|---|---|
| Group | Treatment | Double Initial Volume | in Doubling Time |
| 1 | Control | 5.6 ± 0.4 | — |
| 2 | Radiation only | 8.3 ± 1.7 | 2.7 ± 1.6 |
| 3 | PDT only | 8.9 ± 1.7 | 3.2 ± 1.7 |
| 4 | Radiation then | 11.0 ± 1.5 | 5.4 ± 1.4 |
| | PDT | | |
| 5 | PDT and Radiation | 13.7 ± 1.6 | 8.1 ± 1.5 |

TABLE 2
p-values Between Different Treatment Groups

| Difference Test | p-value |
|---|---|
| Groups 2 vs. 3 | 0.77 |
| Groups 2 vs. 5 | 0.12 |
| Groups 3 vs. 5 | 0.0013 |
| Groups 4 vs. 5 | 0.049 |

### Example 5

### Comparison of Tumor Oxygen Measurements at Different Irradiances and Dose Rates

Aminolevulinic Acid-Induced Protoporphyrin IX and Verteporfin

Tumor oxygen changes were measured in mice treated either with aminolevulinic acid-induced protoporphyrin IX (ALA-PPIX) or verteporfin. Using electron paramagnetic resonance (EPR) oximetry described in Example 1, two irradiance levels were performed three hours post-injection of verteporfin or ALA-PPIX. Irradiance levels for ALA-PPIX were 30 mW/cm² and 150 mW/cm², whereas 50 mW/cm² and 200 mW/cm² were used for verteporfin.

EPR oximetry measured tumor oxygenation in RIF-1 tumor cells and in tumorigenic mice treated with photodynamic therapy, using synthetic lithium phthalocyanine (LiPc) as the oxygen-sensitive EPR material. Small particles between 50 and 200 μm in size were implanted into the animal tumors by 23-gauge needle at a depth of 1-3 mm within the tumor. Injections of LiPc were performed at least 24 hours prior to photodynamic therapy. Mice were placed in an L-band (1.2 GHz) EPR spectrometer with a microwave bridge that was custom-built for small animal experiments involving measurement of tissue pO₂. Each mouse was anesthetized and placed in the EPR resonator for continuous measurement of the tumor tissue pO₂ in 15-minute intervals. The external loop resonator was positioned over the tumor to record the EPR signal of injected LiPc within the tissue. Line widths of the EPR signal were converted to pO₂ using a calibration curve determined for LiPc. The data were fit with a software program to match the Voigt line shape after the line widths were graphed as a function of time before, during, and after treatment.

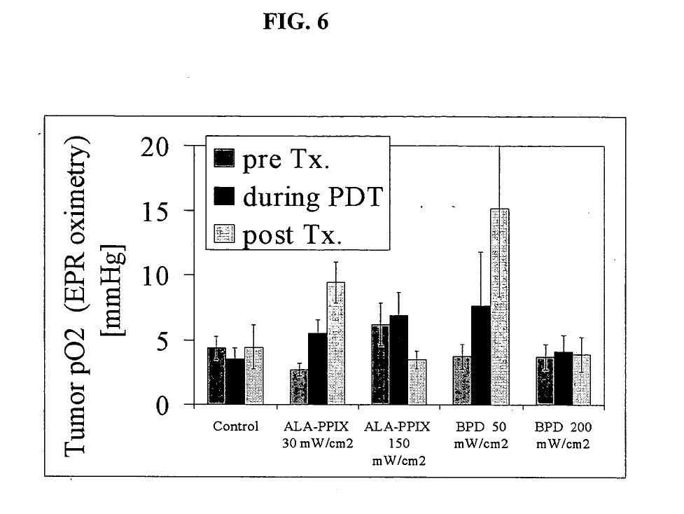

FIG. 6 shows oxygen measurements completed using EPR oximetry on groups of treated and control animals. The error bars show the average values obtained during the first 2 minutes prior to light irradiation, average values obtained for the duration of treatment, and average values for the 2 minutes immediately following light treatment. Oxygen levels did not decrease at all during treatment and oxygen concentrations rose significantly during and after treatment, under conditions where low irradiance levels were used. Tumor pO₂ levels either remained the same in control and verteporfin treated mice receiving high irradiance levels; or remained the same for both ALA-PPIX and verteporfin-treated mice receiving low irradiance levels. At high fluence, ALA-PPIX-treated mice displayed a measurable drop in oxygen, but did not change significantly during treatment. Maximal tumor oxygenation was observed immediately after treatment with non-vascular targeting therapy, using either verteporfin of ALA-PPIX in the RIF-1 tumor cell model. This suggests that increased oxygenation or oxygen re-distribution during and after therapy was significant enough to yield synergistic effects when used in combination with radiation therapy.

Changes in Tissue pO₂ in ALA-PPIX Treated Subjects by Eppendorf Electrode Measurements

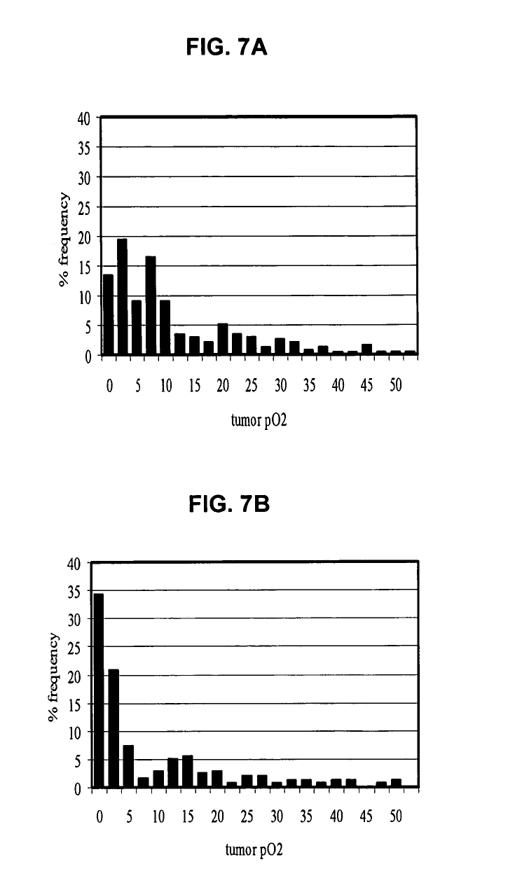

To independently confirm the results described above in ALA-PPIX-treated animals, Eppendorf pO₂ electrode measurements were performed in the same tumor model. Two sections of animals with tumors were irradiated for 45 minutes. The first group of animals received ALA-injections 3 hours prior to light irradiation at a concentration of 100 mg/kg, and the second group received light alone. The light was at a wavelength of 633 nm, at a fluence of 150 mW/cm². Measurements were taken from the tumors immediately after the light was turned off. As depicted in FIGS. 7A and 7B, Eppendorf electrode measurements were displayed as standard histograms of pO₂ expressed in units of mm Hg) within the tumors measured and graphed as a function of the frequency percentage of each pO₂ value occurring. Collected data were pooled from three tracks in each animal with seven control animals (FIG. 7A) and 6 treated animals (FIG. 7B). The median pO₂ for the control group was 3.7 mm Hg, while the median for the treated group was 8.7 mm Hg. Clearly, significant increases in tumor oxygenation occurred after treatment. It is difficult to directly compare values obtained with EPR oximetry and Eppendorf electrode measurements, however both methods independently displayed more than two-fold increases in tumor oxygenation from a highly hypoxic level.

### REFERENCES

1. Weishaupt, K., Gomer, C., and Dougherty, T. Identification of singlet oxygen as the cytotoxic agent in photo-inactivation of a murine tumor. Cancer Res., 36: 2326-2329, 1976.
2. Gibson, S. L. and Hilf, R. Interdependence of fluence, drug dose and oxygen on HPD induced photosensitization of tumor mitochondria. Photochem. Photobiol., 42: 367-373, 1985.
3. Henderson, B. W. and Dougherty, T. J. How does photodynamic therapy work? Photochem. Photobiol., 55: 145-157, 1992.
4. Dougherty, T. J., Gomer, C. J., Henderson, B. W., Jori, G., Kessel, D., Korbelik, M., Moan, J., and Peng, Q. Photodynamic therapy. J. Nat. Cancer Inst., 90: 889-905, 1998.
5. Luksiene, Z., Kalvelyte, A., and Supino, R. On the combination of photodynamic therapy with ionizing radiation. Journal of Photochemistry & Photobiology. B—Biology, 52: 35-42, 1999.
6. Allman, R., Cowburn, P., and Mason, M. Effect of photodynamic therapy in combination with ionizing radiation on human squamous cell carcinoma cell lines of the head and neck. British Journal of Cancer, 83: 655-661, 2000.
7. Scott, L. J. and Goa, K. L. Verteporfin. Drugs & Aging, 16: 139-146; discussion 147-138, 2000.
8. Pogue, B. W., O'Hara, J. A., Goodwin, I. A., Wilmot, C. J., Fournier, G. P., Akay, A. R., and Swartz, H. M. Tumor pO2 Changes during Photodynamic Therapy Depend upon Photosensitizer Type and Time After Injection. Comp. Biochem. Physiol. Part A, 132: 172-184, 2002.
9. Kostron, H. The interaction of hematoporphyrin derivative, light and ionizing radiation in a rat glioma model. Cancer, 57: 964-970, 1986.
10. Moan, J. and Petersen, E. O. X-irradiation of human cells inculture in the presence of hematoporphyrin. Int. J. Radiat. Biol., 50: 107-109, 1981.
11. Bellnier, D. and Dougherty, T. J. Hematoporphyrin derivative photosensitization and gamma-radiation damage interaction in Chinese hampster ovary fibroblasts. Int. J. Rad. Biol., 50: 659-664, 1986.
12. Roberts, D. J. H., Cairnduff, F., Dixon, B., and Brown, S. B. The Response of a Rodent Fibrosarcoma to Combined Treatment with Photodynamic Therapy and Radiotherapy. Int J Oncol, 6: 197-202, 1995.
13. Ramakrishnan, N., Clay, M. E., Friedman, L. R., Antunez, A. R., and Oleinick, N. L. Post-treatment interactions of photodynamic and radiation-induced cytotoxic lesions. Photochemistry & Photobiology, 52: 555-559, 1990.
14. Dobler-Girdziunaite, D., Burkard, W., Haller, U., Larsson, B., and Walt, H. [The combined use of photodynamic therapy with ionizing radiation on breast carcinoma cells in vitro]. Strahlentherapie und Onkologie, 171: 622-629, 1995.
15. Kukielczak, B., Romanowska, B., and Bryk, J. Gamma radiation and MC540 photosensitization of melanoma in the hamster's eye. Melanoma Research, 9: 115-124, 1999.
16. Berg, K., Luksiene, Z., Moan, J., and Ma, L. Combined treatment of ionizing radiation and photosensitization by 5-aminolevulinic acid-induced protoporphyrin IX. Rad. Res., 142: 340-346, 1995.
17. Winter, I., Overgaard, J., and Ehlero, N. The effect on photodynamic therapy alone and in combination with misonidazole or x-rays for management of retinoblastoma like tumor. Photochem. Photobiol., 47: 419-423, 1988.
18. Benstead, K. and Moore, J. V. The effect of combined modality treatment with ionising radiation and TPPS-mediated photodynamic therapy on murine tail skin. British Journal of Cancer, 62: 48-53, 1990.
19. Pogue, B. W., Pitts, J. D., Mycek, M.-A., Sloboda, R. D., Wilmot, C. M., Brandsema, J. A., and O'Hara, J. A. In vivo NADH fluorescence monitoring as an assay for cellular damage in photodynamic therapy. Photochem. Photobiol., 74: 817-824, 2002.
20. Runnels, J. M., Chen, B., Ortel, B., Kato, D., and Hasan, T. BPD-MA-mediated photosensitization in vitro and in vivo: cellular adhesion and B1 integrin expression in ovarian cancer cells. Brit. J. Cancer, 80: 946-953, 1999.
21. Morgan, J. and Oseroff, A. R. Mitochondria-based photodynamic anti-cancer therapy. Advanced Drug Delivery Reviews., 49: 71-86, 2001.
22. Pogue, B. W., Paulsen, K. D., O'Hara, Swartz, H. M. Estimation of Oxygen Distribution in RIF-1 Tumors by Diffusion Model-based Interpretation of Pimonidazole-Hypoxia and Eppendorf Measurements. Rad. Res., 155: 15-25, 2001.
23. Twentyman, P. R., Brown, J. M., Gray, J. W., Franko, A. J., Scoles, M. A., and Kallman, R. F. A new mouse tumor model system (RIF-1) for comparison of endpoint studies. J Natl Canc Inst, 64: 595-604, 1980.
24. Fenton, B. M., Way, B. A. Vascular morphometry of KHT and RIF-1 murine sarcomas. Radiother. Oncol., 28: 57-62, 1993.
25. Busch, T. M., Hahn, S. M., Evans, S. M., and Koch, C. J. Depletion of tumor oxygenation during photodynamic therapy: detection by the hypoxia marker EF3 [2-(2-nitroimidazol-1[H]-yl)-N-(3,3,3-trifluoropropyl)acetamide]. Cancer Research, 60: 2636-2642, 2000.
26. Liu, Y. H., Hawk, R. M., and Ramaprasad, S. In vivo relaxation time measurements on a murine tumor model—prolongation of T1 after photodynamic therapy. Magnetic Resonance Imaging, 13: 251-258, 1995.
27. Goda, F., Bacic, G., O'Hara, J. A., Gallez, B., Swartz, H. M., and Dunn, J. F. The relationship between partial pressure of oxygen and perfusion in two murine tumors after X-ray irradiation: a combined gadopentetate dimeglumine dynamic magnetic resonance imaging and in vivo electron paramagnetic resonance oximetry study. Cancer Res., 56: 3344-3349, 1996.
28. O'Hara, J. A., Goda, F., Demidenko, E., and Swartz, H. M. Effect on regrowth delay in a murine tumor of scheduling split-dose irradiation based on direct pO2 measurements by electron paramagnetic resonance oximetry. Radiation Research, 150: 549-556, 1998.
29. Pogue, B. W., O'Hara, J. A., Liu, K. J., Hasan, T., and Swartz, H. M. Photodynamic treatment of the RIF-1 tumor with verteporfin with online monitoring of tissue oxygen using electron paramagnetic resonance oximetry. Proceedings of SPIE: Laser Tissue Interaction X, 3601: 108-114, 1999.
30. Fingar, V. H., Mang, T. S., and Henderson, B. W. Modification of photodynamic therapy-induced hypoxia by fluosol-DA (20%) and carbogen breathing in mice. Cancer Research, 48: 3350-3354, 1988.
31. Henderson, B. W. and Fingar, V. H. Oxygen limitation of direct tumor cell kill during photodynamic treatment of a murine tumor model. Photochem. Photobiol., 49: 299-304, 1989.
32. Fingar, V. H., Wieman, T. J., Park, Y. J., and Henderson, B. W. Implications of a pre-existing tumor hypoxic fraction on photodynamic therapy. Journal of Surgical Research, 53: 524-528, 1992.
33. Zaidi, S. I. A., Kenney, M. E., and Mukhtar, H. Photodynamic Therapy of RIF-1 Tumors with Topical Application of Silicon Phthalocyanine—Evidence for Induction of Apoptosis During Tumor Ablation. Clin Res, 41: A507-A507, 1993.
34. Bremner, J. C., Bradley, J. K., Adams, G. E., Naylor, M. A., Sansom, J. M., and Stratford, I. J. Comparing the anti-tumor effect of several bioreductive drugs when used in combination with photodynamic therapy (PDT). International Journal of Radiation Oncology, Biology, Physics, 29: 329-332, 1994.
35. van Geel, I. P. J., Oppelaar, H., Marijnissen, J. P. A., Stewart, F. A. Influence of fractionation and fluence rate in photodynamic therapy with photofrin or mTHPC. Radiat. Res., 145: 602-609, 1996.
36. Veenhuizen, R. B. and Stewart, F. A. The Importance of Fluence Rate in Photodynamic Therapy—Is There a Parallel with Ionizing-Radiation Dose-Rate Effects. Radiother Oncol, 37: 131-135, 1995.
37. Tromberg, B. J., Orenstein, A., Kimel, S., Barker, S. J., Hyatt, J., Nelson, J. S., and Berns, M. W. In vivo tumor oxygen tension measurements for the evaluation of the efficiency of photodynamic therapy. Photochem. Photobiol., 52: 375-385, 1990.
38. Tromberg, B. J., Kimel, S., Orenstein, A., Barker, S. J., Hyatt, J., Nelson, J. S., Roberts, W. G., and Berns, M. W. Tumor oxygen tension during photodynamic therapy. J. Photochem. Photobiol. B: Biol., 5: 121-126, 1990.
39. Foster, T. H., Murant, R. S., Bryant, R. G., Knox, R. S., Gibson, S. L., and Hilf, R. Oxygen consumption and diffusion effects in photodynamic therapy. Radiat. Res., 126: 296-303, 1991.
40. Gibson, S. L., VanDerMeid, K. R., Murant, R. S., Raubertas, R. F., and Hilf, R. Effects of various photoradiation regimens on the antitumor efficacy of photodynamic therapy for R3230AC mammary carcinomas. Cancer Res., 50: 7236-7241, 1990.
41. Gibson, S. L., Foster, T. H., Feins, R. H., Raubertas, R. F., Fallon, M. A., and Hilf, R. Effects of Photodynamic Therapy on Xenografts of Human Mesothelioma and Rat Mammary-Carcinoma in Nude-Mice. Brit. J. Cancer, 69: 473-481, 1994.
42. Hua, Z. X., Gibson, S. L., Foster, T. H., and Hilf, R. Effectiveness of Delta-Aminolevulinic Acid-Induced Protoporphyrin as a Photosensitizer for Photodynamic Therapy in-Vivo. Cancer Res., 55: 1723-1731, 1995.
43. Reed, M. W., Wieman, T. J., Schuschke, D. A., Tseng, M. T., and Miller, F. N. A comparison of the effects of photodynamic therapy on normal and tumor blood vessels in the rat microcirculation. Radiat. Res., 119: 542-552, 1989.
44. Fingar, V. H., Wieman, T. J., Wiehle, S. A., and Cerrito, P. B. The role of microvascular damage in photodynamic therapy: the effect of treatment on vessel constriction, permeability, and leukocyte adhesion. Cancer Res., 52: 4914-4921, 1992.
45. Fingar, V. H., Kik, P. K., Haydon, P. S., Cerrito, P. B., Tseng, M., Abang, E., and Wieman, T. J. Analysis of acute vascular damage after photodynamic therapy using benzoporphyrin derivative (BPD). Brit. J. Cancer, 79: 1702-1708, 1999.
46. linuma, S., Schomacker, K. T., Wagnieres, G., Rajadhyaksha, M., Bamberg, M., Momma, T., and Hasan, T. In vivo fluence rate and fractionation effects on tumor response and photobleaching: photodynamic therapy with two photosensitizers in an orthotopic rat tumor model. Cancer Res., 59: 6164-6170, 1999.
47. Major, A., Kimel, S., Mee, S., Milner, T. E., Smithies, D. J., Srinivas, S. M., Chen, Z., Nelson, J. S. Microvascular photodynamic effects determined in vivo using optical doppler tomography. IEEE J. Sel. Top. Quan. Elec., 5: 1168-1175, 1999.
48. Chen, B., Pogue, B. W., Goodwin, I. A., Wilmot, C. M., O'Hara, J., and Hoopes, P. J. Blood flow changes in the RIF-1 tumor in response photodynamic therapy with verteporfin. (submitted to Brit. J. Cancer, 2002).
49. Secomb, T. W., Hsu, R., Ong, E. T., Gross, J. F., and Dewhirst, M. W. Analysis of the effects of oxygen supply and demand on hypoxic fraction in tumors. Acta Oncol., 34: 313-316, 1995.
50. Dewhirst, M. W., Secomb, T. W., Ong, E. T., Hsu, R., and Gross, J. F. Determination of local oxygen consumption rates in tumors. Cancer Res., 54: 3333-3336, 1994.
51. Gulledge, C. J. and Dewhirst, M. W. Tumor oxygenation: a matter of supply and demand. Anticancer Res., 16: 741-749, 1996.
52. Kessel, D., Luo, Y., Deng, Y., and Chang, C. K. The role of subcellular localization in initiation of apoptosis by photodynamic therapy. Photochem. Photobiol., 65: 422-426, 1997.
53. Kessel, D. and Luo, Y. Mitochondrial photodamage and PDT-induced apoptosis. J. Photochem. Photobiol. B: Biol., 42: 89-95, 1998.
54. Kessel, D. and Luo, Y. Photodynamic therapy: a mitochondrial inducer of apoptosis. Cell Death Diff., 6: 28-35, 1999.
55. Oleinick, N. L. and Evans, H. H. The photobiology of photodynamic therapy: cellular targets and mechanisms. Radiat. Res., 150: S146-156, 1998.
56. Xue, L., He, J., and Oleinick, N. L. Promotion of photodynamic therapy-induced apoptosis by stress kinases. Cell Death Diff., 6: 855-864, 1999.
57. Richter, A. M., Waterfield, E., Jain, A. K., Canaan, A. J., Allison, B. A., and Levy, J. G. Liposomal delivery of a photosensitizer, benzoporphyrin derivative monoacid ring A (BPD), to tumor tissue in a mouse tumor model. Photochem. Photobiol., 57: 1000-1006, 1993.
58. Swartz, H. M., Boyer, S., Gast, P., Glockner, J. F., Hu, H., Liu, K. L., Moussavi, M., Norby, S. W., Vahidi, N., Walczak, T., Wu, M., and Clarkson, R. B. Measurements of pertinent concentrations of oxygen in vivo. Mag. Res. Med., 20: 333-339, 1991.
59. Liu, K. J., Gast, P., Moussavi, M., Norby, S. W., Vahidi, N., Walczak, T., Wu, M., and Swartz, H. M. Lithium phthalocyanine: a probe for electron paramagnetic resonance oximetry in viable biological systems. Proc. Nat. Acad. Sci. USA, 90: 5438-5442, 1993.
60. O'Hara, J. A., Goda, F., Liu, K. L., Bacic, G., Hoopes, P. J., and Swartz., H. M. Oxygenation in a murine tumor following radiation: An in vivo electron paramagnetic resonance oximetry study. Radiat. Res., 144: 224-229, 1995.
61. O'Hara, J. A., Goda, F., Liu, K. J., Bacic, G., Hoopes, P. J., and Swartz, H. M. The pO2 in a murine tumor after irradiation: an in vivo electron paramagnetic resonance oximetry study. Radiat. Res., 144: 222-229, 1995.
62. Demidenko, E. Asymptotoic properties of mixed effects models. In: T. G. Gregorie (ed.), Modeling Longitudinal and Spatially Correlated Data. New York: Kluewer Press, 1997.
63. Rice, J. A. Mathematical Statistics and Data Analysis: Belmont: Duxbury Press, 1995.
64. Schmidt-Erfurth, U., Bauman, W., Gragoudas, E., Flotte, T. J., Michaud, N. A., Birngruber, R., and Hasan, T. Photodynamic therapy of experimental choroidal melanoma using lipoprotein-delivered benzoporphyrin. Ophthalmology, 101: 89-99, 1994.
65. Henderson, B. W. and Bellnier, D. A. Tissue localization of photosensitizers and the mechanism of photodynamic tissue destruction. [Review]. Ciba Foundation Symposium, 146: 112-125; discussion 125-130, 1989.
66. Cincotta, L., Szeto, D., Lampros, E., Hasan, T., and Cincotta, A. H. Benzophenothiazine and benzoporphyrin derivative combination phototherapy effectively eradicates large murine sarcomas. Photochem. Photobiol., 63: 229-237, 1996.
67. Henderson, B. W., Busch, T. M., Vaughan, L. A., Frawley, N. P., Babich, D., Sosa, T. A., Zollo, J. D., Dee, A. S., Cooper, M. T., Bellnier, D. A., Greco, W. R., and Oseroff, A. R. Photofrin photodynamic therapy can significantly deplete or preserve oxygenation in human basal cell carcinomas during treatment, depending on fluence rate. Cancer Research., 60: 525-529, 2000.
68. Berg, K., Steen, H. B., Winkelman, J. W., and Moan, J. Synergistic effects of photoactivated tetra(4-sulfonatophenyl)porphine and nocodazole on microtubule assembly, accumulation of cells in mitosis and cell survival. Journal of Photochemistry & Photobiology, B—Biology. 13: 59-70, 1992.
69. Castro, D. J., Saxton, R. E., Haghighat, S., Reisler, E., Plant, D., and Soudant, J. The synergistic effects of rhodamine-123 and merocyanine-540 laser dyes on human tumor cell lines: a new approach to laser phototherapy. Otolaryngol Head Neck Surg, 108: 233-242, 1993.
70. Dewhirst, M. W., Sim, D. A., Gross, J. F., and Kundrat, M. A. Effects of heating rate on normal and tumor microcirculatory function. In: R. B. R. K. R. Diller (ed.), Heat and Mass Transfer in the Microcirculation of Thermally Significant Vessels, pp. 75-80: ASME, 1986.
71. Goff et al. Cancer Res. 51:4762-4767; 1991.
72. Hamblin et al. Cancer Res. 61:7155-7162; 2001.
74. Jiang et al. J. Immunol. Meth. 134:139-149; 1990.
75. Richter, A. M., Kelly, B., Chow, J., Liu, D. J., Towers, G. H. N., Dolphin, D., and Levy, J. G. (1987) Preliminary studies on a more effective phototoxic agent than hematoporphyrin, JNCI 79, 1327-1331.
76. Aveline, B., Hasan, T., and Redmond, R. W. (1994) Photophysical and photosensitizing properties of benzoporphyrin derivative monoacid ring A (BPD-MA), Photochem Photobiol 59, 328-35.
77. Levy, J. G. (1994) Photosensitizers in photodynamic therapy, Semin Oncol 21, 4-10.
78. Savellano, M. D. (2000) Photodynamic Targeting with Photosensitizer Immunoconjugates., PhD Thesis, Department of Biomedical Engineering, University of Michigan, UMI Dissertations Publishing,
79. Hamblin, M. R., Miller, J. L., and Hasan, T. (1996) Effect of charge on the interaction of site-specific photoimmunoconjugates with human ovarian cancer cells, Cancer Res 56, 5205-10.
80. Waksal, H. W. (1999) Role of an anti-epidermal growth factor receptor in treating cancer, Cancer Metastasis Rev 18, 427-36.
81. Fletcher, M. and Goldstein, A. L. (1987) Recent advances in the understanding of the biochemistry and clinical pharmacology of interleukin-2. Lymphokine Res 6, 45-57.
82. Rabinowich, H., Cohen, R., Bruderman, I., Z., S., and Klajman, A. (1987) Functional analysis of mononuclear cells infiltrating into tumors: lysis of autologous human tumor cells by cultured infiltrating lymphocytes., Cancer Res 47, 173-7.
83. Rosenberg, S. A., Spiess, P., and Lafreniere, R. (1986) A new approach to the adoptive immunotherapy of cancer with tumor-infiltrating lymphocytes, Science 233, 1318-21. (Rabinowich et al., 1987).
84. Pizza, G., Severini, G., Menniti, D., De Vinci, C., and Corrado, F. (1984) Tumour regression after intralesional injection of interleukin 2 (IL-2) in bladder cancer. Preliminary report, Int J Cancer 34, 359-67.
85. Sperduto, P. W., DeLaney, T. F., Thomas, G., Smith, P., Dachowski, L. J., Russo, A., Bonner, R., and Glatstein, E. (1991) Photodynamic therapy for chest wall recurrence in breast cancer, Int J Radiat Oncol Biol Phys 21, 441-6.
86. Walther, M. M., Delaney, T. F., Smith, P. D., Friauf, W. S., Thomas, G. F., Shawker, T. H., Vargas, M. P., Choyke, P. L., Linehan, W. M., Abraham, E. H., et al. (1997) Phase I trial of photodynamic therapy in the treatment of recurrent superficial transitional cell carcinoma of the bladder, Urology 50, 199-206.
87. Neuwelt, E. A., and Rapoport, S. I. (1984) Modification of the blood-brain barrier in the chemotherapy of malignant brain tumors., Fed Proc 43, 214-9.
88. Baba, T., Black, K. L., Ikezaki, K., Chen, K. N., and Becker, D. P. (1991) Intracarotid infusion of leukotriene C4 selectively increases blood-brain barrier permeability after focal ischemia in rats., J Cereb Blood Flow Metab 11, 638-43.
89. Gennuso, R., Spigelman, M. K., Chinol, M., Zappulla, R. A., Nieves, J., Vallabhajosula, S., Alberto Paciucci P, Goldsmith, S. J. a., and Holland, J. F. (1993) Effect of blood-brain barrier and blood-tumor barrier modification on central nervous system liposomal uptake, Cancer Invest 11, 118-28.

Having thus described in detail preferred embodiments of the present invention, it is to be understood that the invention defined by the appended claims is not to be limited to particular details set forth in the above description, as many apparent variations thereof are possible without departing from the spirit or scope of the present invention. Modifications and variations of the method and apparatuses described herein will be obvious to those skilled in the art, and are intended to be encompassed by the following claims.

1. A method for treating a tumor in a subject, said method comprising the steps of:
(a) administering a photosensitizer to the subject;
(b) following a period of time after step (a), irradiating the tumor such that cellular metabolism is decreased and oxygen is distributed to hypoxic areas of the tumor; and
(c) administering radiation therapy thereafter to thereby treat the tumor.

2. The method of claim 1, wherein the period of time ranges from about 30 minutes to about 48 hours.

3. The method of claim 1, further comprising obtaining the photosensitizer.

4. The method of claim 1, wherein the tumor is in the mouth, esophagus, stomach, small intestine, large intestine, trachea, larynx, lung, cervix, uterus, ovary, prostate, testicles or brain of the subject.

5. The method of claim 1, wherein the tumor is on the skin or not more than about 3 centimeters under the skin of the subject.

6. The method according to claim 1, wherein the photosensitizer is localized in the mitochondrial membrane of the cells comprising the tumor.

7. The method according to claims 1, wherein the photosensitizer is cationic.

8. The method of claim 1, wherein the photosensitizer is verteporfin or aminolevulinic acid.

9. The method of claim 1, wherein the photosensitizer is coupled to a targeting moiety.

10. The method of claim 9, wherein the targeting moiety is an antibody.

11. The method of claim 1, wherein the photosensitizer is administered topically or systemically.

12. The method of claim 11, wherein systemic administration of the photosensitizer is intravenous.

13. The method of claim 1, wherein the tumor is irradiated about three hours after step (a).

14. The method according to claim 1, wherein irradiation is administered between about 1 to about 5 hours after administering the photosensitizer.

15. The method according to claim 1, wherein irradiation is administered between about 1 to about 3 hours after administering the photosensitizer.

16. The method of claim 1, wherein irradiation is performed with a laser.

17. The method of claim 1, wherein irradiation is performed with an optical fiber.

18. The method of claim 1, wherein the radiation therapy administered as an x-ray.

19. The method of claim 1, wherein the subject is a human.

20. A method for treating a tumor in a subject, said method comprising the steps of:
(a) administering a photosensitizer to the subject;
(b) following a period of time after step (a), irradiating the tumor such that metabolism is decreased and oxygen is distributed to hypoxic areas of the tumor; and
(c) administering radiation therapy concurrently with or prior to step (b) to thereby treat the tumor.

21. The method of claim 20, wherein the period of time ranges from about 30 minutes to about 48 hours.

22. The method of claim 20, further comprising obtaining the photosensitizer.

23. A method for treating a tumor in a subject, said method comprising the steps of:
(a) administering radiation therapy;
(b) administering a photosensitizer to the subject; and
(c) following a period of time after step (b), irradiating the tumor such that cellular metabolism is decreased and oxygen is distributed to hypoxic areas of the tumor to thereby treat the tumor.

24. The method of claim 23, wherein the period of time ranges from about 30 minutes to about 48 hours.

25. The method of claim 23, further comprising obtaining the photosensitizer.

26. A method for reducing malignant cell proliferation, said method comprising the steps of:
(a) contacting malignant cells with a photosensitizer that localizes to an intracellular compartment following a period of time;
(b) irradiating the malignant cells of step (a) such that cellular metabolism is decreased and oxygen is distributed to hypoxic cells; and
(c) providing radiation to the malignant cells before, after or concurrently with step (b) to thereby decrease malignant cell proliferation.

27. The method of claim 26, wherein the period of time ranges from about 30 minutes to about 48 hours.

28. The method of claim 26, further comprising obtaining the photosensitizer.

29. The method according to claim 26, wherein the photosensitizer is verteporfin or aminolevulinic acid.

30. The method according to claim 26, wherein the intracellular compartment is the mitochondria.

31. A kit for treating a tumor or reducing malignant cell proliferation comprising a photosensitizer and instructions for administering the photosensitizer to a subject in need thereof in accordance with the method of any of claims 1-26.
